(19) United States
(12) Patent Application Publication
(10) Pub. No.: US 2007/0298105 A1
(43) Pub. Date: Dec. 27, 2007

(54) METHODS OF TREATING CONDITIONS BY SUSTAINED RELEASE ADMINISTRATION OF BENZIMIDAZOLE DERIVATIVES

(76) Inventor: Stephen S. Hwang, Palo Alto, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/768,860

(22) Filed: Jun. 26, 2007

Related U.S. Application Data
(60) Provisional application No. 60/817,213, filed on Jun. 27, 2006.

(51) Int. Cl. A61K 31/4439; A61K 9/22; A61K 9/24

(52) U.S. Cl. 424/468; 514/338; 424/473

(57) ABSTRACT

Disclosed are methods including orally administering oral sustained release dosage forms comprising a benzimidazole derivative to a patient suffering from one or more gastric acid related diseases; wherein the benzimidazole derivative is sustainably released from the oral sustained release dosage form for a period of at least about 10 hours, and at rates effective to maintain an intragastric pH of the patient ≧about 4 during at least about 65% of the period. Related compositions are also disclosed.

### CROSS REFERENCE TO RELATED APPLICATIONS

This application claims the benefit of U.S. Provisional Application Ser. No. 60/817,213, filed Jun. 27, 2007.

### FIELD OF THE INVENTION

The invention relates to treatment of gastric acid related diseases, more particularly to treatment of gastric acid related diseases using sustained release administration of benzimidazole derivatives.

### BACKGROUND

Certain conditions and/or diseases are generally known to be associated with abnormalities in the amount, location and/or effect of gastric acid. Such conditions and/or diseases are referred to as gastric acid related diseases. Gastric acid related diseases include a broad spectrum of conditions and/or diseases such as (but not limited to) erosive or ulcerative gastroesophogeal reflux disease; duodenal ulcers; pathological hypersecretory conditions (such as Zollinger-Ellison Syndrome); and acid related extra-esophageal manifestations of gastroesophogeal reflux disease in patients with chronic cough, asthma, chronic obstructive airway disease, pharyngitis, or otitis media.

Drugs useful in treating such gastric acid related diseases include proton pump inhibitors. Proton pump inhibitors act by irreversibly or substantially irreversibly blocking the gastric proton pump of gastric parietal cells. The proton pump is the terminal stage in gastric acid secretion, being directly responsible for secreting H+ ions into the gastric lumen. Proton pump inhibitors may reduce gastric acid secretion by up to 99%; thus promoting resolution of gastric acid related diseases.

However, under certain circumstances, daytime or nocturnal gastric acid breakthrough can occur even when gastric acid pH is being controlled using proton pump inhibitors such as benzimidazole derivatives. Nocturnal gastric acid breakthrough may be defined as the occurrence of an intragastric pH<4 for more than one continuous hour overnight. Nocturnal gastric acid breakthrough can disturb sleep, and can significantly delay resolution of gastric acid related diseases. Similarly, daytime gastric acid breakthrough can create discomfort and significantly delay resolution of gastric acid related diseases.

What is needed is improved methods and dosage forms applicable in treatment of gastric acid related diseases, particularly embodiments that may reduce nocturnal gastric acid breakthrough.

### SUMMARY OF THE INVENTION

In an aspect, the invention relates to a method comprising: orally administering oral sustained release dosage forms comprising a benzimidazole derivative to a patient suffering from one or more gastric acid related diseases; wherein the benzimidazole derivative is sustainably released from the oral sustained release dosage form for a period of at least about 10 hours, and at rates effective to maintain an intragastric pH of the patient ≧about 4 during at least about 65% of the period.

### BRIEF DESCRIPTION OF THE DRAWINGS

FIG. 1 shows an elementary osmotic pump dosage form embodiment of the present invention.

FIG. 2 shows an osmotic dosage form embodiment according to the present invention.

FIG. 3 shows a soft capsule embodiment of an osmotic dosage form according to the invention.

FIG. 4 shows a hard capsule embodiment of an osmotic dosage form according to the invention.

FIGS. 5A-5C show a dosage form according to the invention.

FIG. 6 shows mean plasma concentration-time profiles for rabeprazole and esomeprazole.

FIG. 7 shows mean gastric pH profiles (30 min average) following rabeprazole and esomeprazole treatments (N=19) in Example 4.

FIG. 8 shows mean difference from placebo (30 min average) following rabeprazole and esomeprazole treatments (N=19) in Example 4.

### DETAILED DESCRIPTION

### I. Introduction

The inventors have unexpectedly realized that the problems noted above can be solved by methods comprising: orally administering oral sustained release dosage forms comprising a benzimidazole derivative to a patient suffering from one or more gastric acid related diseases; wherein the benzimidazole derivative is sustainably released from the oral sustained release dosage form for a period of at least about 10 hours, and at rates effective to maintain an intragastric pH of the patient ≧ about 4 during at least about 65% of the period, and by related dosage forms.

It is generally recognized that the therapeutic effect of interest for proton pump inhibitors is the inhibition of gastric acid secretion. This effect can be measured by several different parameters and one commonly used parameter is the % time gastric pH>4 or its compliment, % time gastric pH<4. In particular, the inventors have noted that divided doses of benzimidazole derivatives can provide improved therapeutic benefits as compared to single daily doses for certain patient populations. This is described in more detail in M. Sugimoto et al., “Different dosage regimens of rabeprazole for nocturnal gastric acid inhibition in relation to cytochrome P450 2C19 genotype status.” Clin. Pharmacology & Therapeutics 76(4):290-301(2004) (“Sugimoto”).

Sugimoto notes that, for the treatment of gastroesophageal reflux disease, intragastric pH should be lower than 4.0 for no more than 4 hours a day. Under certain circumstances, nocturnal gastric acid breakthrough can occur even when gastric acid pH is being controlled using PPI's such as benzimidazole derivatives. Sugimoto then provided data that suggested that dividing the dose of a benzimidazole derivative can provide decreased % time with gastric pH <4.0 both at night (reduced nocturnal gastric acid breakthrough) and during the day for certain patients. These patients include homozygous and heterozygous extensive metabolizers of benzimidazole derivatives. Such patients make up the majority of patients that are typically treated for gastric acid related diseases. The incidence of poor metabolizer ranges from 13 to 23% in Asians, 1 to 6% in Caucasians and 1 to 7.5% in black Africans as reviewed by Desta et al, “Clinical Significance of the Cytochrome P450 2C19 Genetic Polymorphism.” Clinical Pharmacokinetics 41(12) 913-58 (2002) (“Desta”). The benefits seen in divided dosing in gastroesophageal reflux disease might be expected to extend to gastric acid related diseases generally, because in both cases, the tissue damage is induced by the presence of excess gastric acid.

Dosing in divided doses is inconvenient, however, and patient compliance with such a regimen may be low. Low compliance can lead to less effective inhibition of gastric acid secretion, and consequently poor gastric acid control. Poor gastric acid control may result in nocturnal or daytime gastric acid breakthrough, which would lead to gastric acid related diseases and slower healing of the lesions defeat the purpose of divided dosing.

Accordingly, the inventors realized that providing an oral sustained release dosage form suitable for sustainably releasable dosing of benzimidazole derivatives would provide a similar benefit to the divided dose strategy of Sugimoto, while simultaneously helping to improve patient compliance. Therefore, an advantage of such oral sustained release dosage forms, and related methods, could be enhanced inhibition of gastric acid secretion as measured by reduced the % of time gastric pH <4.0 including reduced nocturnal or daytime gastric acid breakthrough. The extent of oesophageal mucosal injury is determined by the degree and duration of oesophageal acid exposure, which, in turn, is related to the pH of the gastric contents. The effect of antisecretory therapy on GORD/GERD (gastro-oesophageal reflux disease) can be predicted from the duration of suppression of intragastric acidity to above pH 4.0 achieved by each drug regimen, and the length of treatment. This has been stated by Bell et al., “Appropriate Acid Suppression for the management of gastro-oesophageal reflux disease.” In Digestion 51 (Suppl 1) 59-67 (1992).

However, prior to the present invention it was not known if useful oral sustained release dosage forms that comprised benzimidazole derivatives could be developed for treatment of gastric acid related indications. This is because, prior to the present invention, it was not known whether benzimidazole derivatives could be absorbed from the colon. Drugs having poor colonic absorption are absorbed by the body through a period of only four to six hours after oral ingestion, because the typical residence time of a drug in the upper G.I. tract is from approximately four to six hours. To achieve an oral sustained release dosage form according to the invention, it would be necessary for benzimidazole derivatives to be absorbed for a longer period. Such a longer period would necessarily include time when the inventive oral sustained release dosage form would reside in the colon. If the benzimidazole derivatives were not colonically absorbed at a therapeutically meaningful level, then use of a sustained release strategy might not be successful. Instead, the benzimidazole derivatives released by the oral sustained release dosage form after the dosage form entered the colon would not be absorbed and, instead, would be expelled from the body.

Accordingly, the inventors investigated the colonic absorption of benzimidazole derivatives. They discovered surprisingly that the benzimidazole derivatives were colonically absorbed at a therapeutically meaningful level.

The results can be seen in Example 4. Rabeprazole 60 mg colonic treatment achieved similar area under the curve (AUC) as that of 20 mg oral tablet. The colonic bioavailability of rabeprazole was estimated to be approximately 40% compared to that of the oral tablets on a dose-normalized basis. Rabeprazole mean Cmax and AUC increased approximately dose proportionally following the 30 mg and 60 mg colonic treatments. Additionally, and further surprisingly, regardless of the similar AUC achieved from the rabeprazole 60 mg colonic treatment and 20 mg oral tablet, rabeprazole 60 mg colonic treatment provided a better gastric pH control than rabeprazole 20 mg tablet, especially during the night-time period.

For instance, while the effects on gastric pH were comparable between rabeprazole 60 mg colonic treatment and rabeprazole 20 mg oral tablet during the time period from 0 to 12 hr, the mean effects were significantly higher with the 60 mg colonic treatment than that of 20 mg tablet in the subsequent time periods (12-30 hr, Tables C and D). This may have been due to higher plasma concentrations achieved by the 60 mg colonic treatment during the later time periods. The data indicates that, in this study, rabeprazole 60 mg colonic treatment provided a better gastric pH control than rabeprazole 20 mg tablet, especially during the night-time period.

The results of Example 4 suggest that benzimidazole derivatives can be colonically absorbed at levels sufficient to provide a measureable pharmacological effect (i.e. gastric acid suppression). This supports the novelty and unobviousness of the inventive methods and dosage forms.

The invention, and embodiments thereof, will now be described in more detail.

### II. Definitions

All percentages are weight percent unless otherwise noted.

All references cited herein are incorporated herein by reference in their entirety and for all purposes to the same extent as if each individual publication or patent or patent application was specifically and individually indicated to be incorporated by reference in its entirety for all purposes. The discussion of references herein is intended merely to summarize the assertions made by their authors and no admission is made that any reference constitutes prior art. Applicants reserve the right to challenge the accuracy and pertinence of the cited references.

The present invention is best understood by reference to the following definitions, the drawings and exemplary disclosure provided herein.

“Administering” or “administration” means providing a drug to a patient in a manner that is pharmacologically useful.

“Benzimidazole derivative” means the compound known as 2-[[[4-(3-methoxypropoxy)-3-methyl-2-pyridinyl]-methyl]sulfinyl]-1H-benzimidazole (“rabeprazole”), together with prodrugs of rabeprazole, rabeprazole sodium, and other pharmaceutically acceptable salts of rabeprazole and/or prodrugs thereof.

“Dosage form” means one or more compounds in a medium, carrier, vehicle, or device suitable for administration to a patient. “Oral dosage form” means a dosage form suitable for oral administration.

“Effective amount” means that amount of compound that elicits the biological or medicinal response in a tissue system, animal or human, that is being sought by a researcher, veterinarian, medical doctor, or other clinician, which includes therapeutic alleviation of the symptoms of the disease or disorder being treated and prophylactic. The effective amount of a compound selected from Formula (I) or Formula (II) or pharmaceutical composition thereof may be from about 0.01 mg/Kg/dose to about 300 mg/Kg/dose. Effective amounts may also be from about 0.01 mg/Kg/dose to about 100 mg/Kg/dose. An effective amount also contemplated may be from about 0.05 mg/Kg/dose to about 10 mg/Kg/dose. Another effective amount includes from about 0.1 mg/Kg/dose to about 5 mg/Kg/dose. Therefore, the effective amount of the active ingredient contained per dosage unit as described herein may be in a range of from about 700 ng/dose to about 21 g/dose for a subject having a weight of about 70 Kg. oral dosage form.

“Gastric acid related indications” means those conditions and/or diseases that are associated with abnormalities in the amount, location and/or effect of gastric acid. Preferred gastric acid related indications comprise healing and/or maintenance of healing of erosive or ulcerative gastroesophogeal reflux disease; healing of duodenal ulcers; treatment of pathological hypersecretory conditions; treatment of symptiomatic gastroesophogeal reflux disease (GERD), treatment of acid related extra esophageal manifestations of GERD in patients with chronic cough, asthma, COPD, pharyngitis, or otitis media, and/or Heliobacter Pylori eradication to reduce risk of duodenal ulcer recurrence. Preferred pathological hypersecretory conditions comprise Zollinger-Ellison Syndrome.

“Intragastric pH” means the stomach fluid pH as measured by a pH electrode placed in a healthy volunteer/patient's stomach. In preferred embodiments, methods according to the invention comprise releasing one or more benzimidazole derivatives at rate(s) effective to maintain an intragastric pH of the patient ≧about 4 during at least about 65% of the period, more preferably at least 70% of the period, still more preferably at least 75% of the period, and yet more preferably at least 80% of the period.

“Medicament” means a product for use in preventing, treating or ameliorating substance related disorders such as substance dependence, substance abuse or substance induced disorders in a subject in need thereof.

“Oral sustained release dosage form” means a dosage form suitable for oral administration to a patient comprising one or more compounds, wherein the dosage form operates to sustainably release the one or more compounds. In an embodiment, the oral sustained release dosage form is adapted to sustainably release the one or more compounds (preferable one or more benzimidazole derivatives) over a period of greater than about 4 hours, more preferably greater than about 8 hours, more preferably greater than about 10 hours, more preferably still greater than about 14 hours, even more preferably greater than about 16 hours and up to about 24 hours.

“Orally administering” or “oral administration means administering or administration by mouth.

“Osmotic oral sustained release dosage form” means an oral sustained release dosage form wherein the dosage form operates via an osmotic mechanism to sustainably release one or more compounds (preferable one or more benzimidazole derivatives).

“Patient” means an animal, preferably a mammal, more preferably a human, in need of therapeutic intervention or under study, such as a healthy volunteer.

“Prolonged period of time” means a continuous period of time of greater than about 2 hours, preferably, greater than about 4 hours, more preferably, greater than about 8 hours, more preferably greater than about 10 hours, more preferably still, greater than about 14 hours, most preferably, greater than about 14 hours and up to about 24 hours.

“Rate of release” or “release rate” or “R” means the quantity of benzimidazole derivative released from a dosage form per unit time, e.g., milligrams of drug released per hour (mg/hr). R may vary as a function of time, or may be a constant, particularly over a defined period. For instance, drug release rates for oral dosage forms of the present invention are measured as an in vitro rate of drug release, i.e., a quantity of drug released from the dosage form per unit time measured under appropriate conditions and in a suitable fluid. R, either constant or varying, may be chosen to provide a specific pharmacokinetic or pharmacodynamic effect, such as Cmax or AUCinf.

The release rates referred to herein may be determined by placing a dosage form to be tested in de-ionized water in metal coil or metal cage sample holders attached to a USP Type VII bath indexer in a constant temperature water bath at 37° C. Aliquots of the release rate solutions, collected at pre-set intervals, are then injected into a chromatographic system fitted with an ultraviolet or refractive index detector to quantify the amounts of drug released during the testing intervals.

The time at which a specified percentage of the drug within a dosage form has been released from said dosage form may be referred to as the “Tx” value, where “x” is the percent of drug that has been released. For example, a commonly used reference measurement for evaluating drug release from dosage forms is the time at which 70% of drug within the dosage form has been released. This measurement is referred to as the “T70” for the dosage form.

“Suffering from” means that a patient presents with a condition and/or disease or has been diagnosed with a condition and/or disease.

“Sustained release” or “sustainably releasing” means release or releasing of a drug or a dose of a drug over a prolonged period of time. In preferred embodiments, such sustained release may take place in a continuous or a pulsed fashion. In certain embodiments, the benzimidazole derivative is sustainably released from the oral sustained release dosage form for a period of at least about 10 hours, preferably for a period of at least about 12 hours, more preferably for a period of at least about 14 hours, still more preferably for a period of at least about 16 hours, even more preferably for a period of at least about 17 hours, and yet more preferably for a period of at least about 18 hours.

“Therapeutically effective amount” means that amount of drug that elicits the biological or medicinal response in a tissue system, animal or human that is being sought by a researcher, veterinarian, medical doctor or other clinician, which includes alleviation of the symptoms of the disease or disorder being treated.

### III. Dosage Forms

In embodiments, the inventive sustained release dosage forms are formulated into dosage forms administrable to patients in need thereof. Sustained release dosage forms and methods of treatment using the sustained release dosage forms will now be described. It will be appreciated that the sustained release dosage forms described below are merely exemplary.

A variety of sustained release dosage forms are suitable for use in the present invention. In certain embodiments, the dosage form is orally administrable and is sized and shaped as a conventional tablet or capsule. Orally administrable dosage forms may be manufactured according to one of various different approaches. For example, the dosage form may be manufactured as a diffusion system, such as a reservoir device or matrix device, a dissolution system, such as encapsulated dissolution systems (including, for example, “tiny time pills”, and beads) and matrix dissolution systems, and combination diffusion/dissolution systems and ion-exchange resin systems, as described in Pharmaceutical Sciences, Remington, 18th Ed., pp. 1676-1686 (1990), Mack Publishing Co.; The Pharmaceutical and Clinical Pharmacokinetics, 3rd Ed., pp. 1-28 (1984), Lea and Febreger, Philadelphia.

Osmotic dosage forms in general utilize osmotic pressure to generate a driving force for imbibing fluid into a compartment formed, at least in part, by a semipermeable membrane that permits free diffusion of fluid but not drug or osmotic agent(s), if present. A significant advantage to osmotic systems is that operation is pH-independent and thus continues at the osmotically determined rate throughout an extended time period even as the dosage form transits the gastrointestinal tract and encounters differing microenvironments having significantly different pH values. A review of such dosage forms is found in Santus and Baker, “Osmotic drug delivery: a review of the patent literature,” Journal of Controlled Release 35 (1995) 1-21, incorporated by reference herein. U.S. Pat. Nos. 3,845,770; 3,916,899; 3,995,631; 4,008,719; 4,111,202; 4,160,020; 4,327,725; 4,578,075; 4,681,583; 5,019,397; and 5,156,850 disclose osmotic devices for the continuous dispensing of active agent.

Osmotic dosage forms in which a drug composition is delivered as a slurry, suspension or solution from a small exit orifice by the action of an expandable layer are disclosed in U.S. Pat. Nos. 5,633,011; 5,190,765; 5,252,338; 5,620,705; 4,931,285; 5,006,346; 5,024,842; and 5,160,743, which are incorporated herein by reference. Typical devices include an expandable push layer and a drug layer surrounded by a semipermeable membrane. In certain instances, the drug layer is provided with a subcoat to delay release of the drug composition to the environment of use or to form an annealed coating in conjunction with the semipermeable membrane.

An exemplary dosage form, referred to in the art as an elementary osmotic pump dosage form, is shown in FIG. 1. Dosage form 20, shown in a cutaway view, is also referred to as an elementary osmotic pump (EOP), and is comprised of a semi-permeable membrane 22 that surrounds and encloses an internal compartment 24. The internal compartment contains a single component layer referred to herein as a drug layer 26, comprising an inventive substance 28 in an admixture with selected excipients. The excipients are adapted to provide an osmotic activity gradient for attracting fluid from an external environment through membrane 22 and for forming a deliverable complex formulation upon imbibition of fluid. The excipients may include a suitable suspending agent, also referred to herein as drug carrier 30, a binder 32, a lubricant 34, and an osmotically active agent referred to as an osmagent 36. Exemplary materials useful for these components can be found disclosed throughout the present application.

Semi-permeable membrane 22 of the osmotic dosage form is permeable to the passage of an external fluid, such as water and biological fluids, but is substantially impermeable to the passage of components in the internal compartment. Materials useful for forming the membrane are essentially nonerodible and are substantially insoluble in biological fluids during the life of the dosage form. Representative polymers for forming the semi-permeable membrane include homopolymers and copolymers, such as, cellulose esters, cellulose ethers, and cellulose ester-ethers. Flux-regulating agents can be admixed with the membrane-forming material to modulate the fluid permeability of the membrane. For example, agents that produce a marked increase in permeability to fluid such as water are often essentially hydrophilic, while those that produce a marked permeability decrease to water are essentially hydrophobic. Exemplary flux regulating agents include polyhydric alcohols, polyalkylene glycols, polyalkylenediols, polyesters of alkylene glycols, and the like.

In operation, the osmotic gradient across membrane 22 due to the presence of osmotically-active agents causes gastric fluid to be imbibed through the membrane, swelling of the drug layer, and formation of a deliverable complex formulation (e.g., a solution, suspension, slurry or other flowable composition) within the internal compartment. The deliverable inventive substance formulation is released through an exit 38 as fluid continues to enter the internal compartment. Even as drug formulation is released from the dosage form, fluid continues to be drawn into the internal compartment, thereby driving continued release. In this manner, the inventive substance is released in a sustained and continuous manner over a prolonged period.

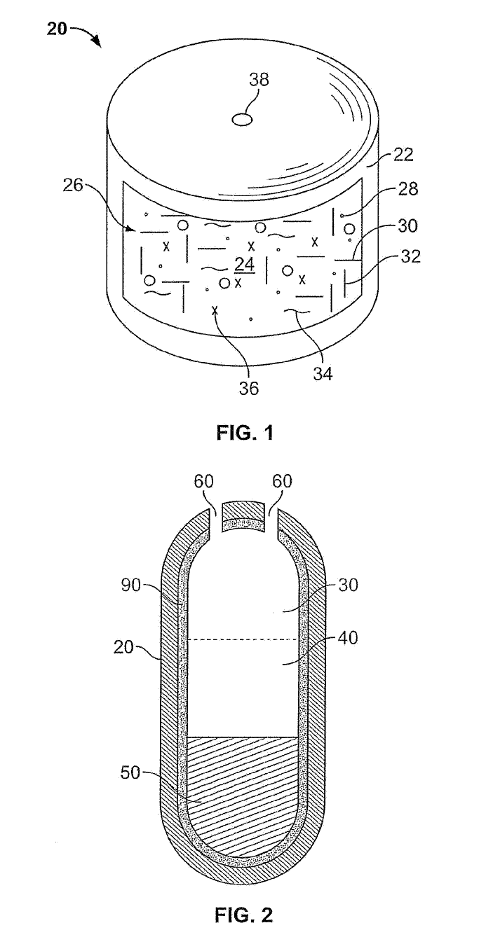

FIG. 2 illustrates certain inventive embodiments of sustained release dosage forms. Dosage forms of this type are described in detail in U.S. Pat. Nos. 4,612,008; 5,082,668; and 5,091,190; and are further described below.

FIG. 2 shows an embodiment of one type of sustained release dosage form, namely the osmotic sustained release dosage form. First drug layer 30 comprises osmotically active components, and a lower amount of benzimidazole derivative than in second drug layer 40. The osmotically active component(s) in the first component drug layer comprises an osmagent such as salt and one or more osmopolymer(s) having relatively small molecular weights which exhibit swelling as fluid is imbibed such that release of these osmopolymers through exit 60 occurs similar to that of drug layer 40. Additional excipients such as binders, lubricants, antioxidants and colorants may also be included in first drug layer 30.

Second drug layer 40 comprises benzimidazole derivative in an admixture with selected excipients adapted to provide an osmotic activity gradient for driving fluid from an external environment through membrane 20 and for forming a deliverable drug formulation upon imbibition of fluid. The excipients may include a suitable suspending agent, also referred to herein as a drug carrier, but no osmotically active agent, “osmagent,” such as salt, sodium chloride. It has been discovered that the omission of salt from this second drug layer, which contains a higher proportion of the overall drug in the dosage form, in combination with the salt in the first drug layer, provides an improved ascending rate of release creating a longer duration of ascending rate.

Drug layer 40 has a higher concentration of benzimidazole derivative than does drug layer 30. The ratio of the concentration of benzimidazole derivative in the first drug layer 30 to the concentration of benzimidazole derivative in the second drug layer 40 is preferably maintained at less than 1 and preferably less than or equal to about 0.43 to provide the desired substantially ascending rate of release.

Drug layer 40 may also comprise other excipients such as lubricants, binders, etc.

Drug layer 40, as with drug layer 30, further comprises a hydrophilic polymer carrier. The hydrophilic polymer contributes to the controlled delivery of the benzimidazole derivative. Representative examples of these polymers are poly(alkylene oxide) of 100,000 to 750,000 number-average molecular weight, including poly(ethylene oxide), poly(methylene oxide), poly(butylene oxide) and poly(hexylene oxide); and a poly(carboxymethylcellulose) of 40,000 to 400,000 number-average molecular weight, represented by poly(alkali carboxymethylcellulose), poly(sodium carboxymethylcellulose), poly(potassium carboxymethylcellulose) and poly(lithium carboxymethylcellulose). Drug layer 40 can further comprise a hydroxypropylalkylcellulose of 9,200 to 125,000 number-average molecular weight for enhancing the delivery properties of the dosage form as represented by hydroxypropylethylcellulose, hydroxypropylmethylcellulose, hydroxypropylbutylcellulose and hydroxypropylpentylcellulose; and a poly(vinylpyrrolidone) of 7,000 to 75,000 number-average molecular weight for enhancing the flow properties of the dosage form. Preferred among these polymers are the poly(ethylene oxide) of 100,000-300,000 number average molecular weight. Carriers that erode in the gastric environment, i.e., bioerodible carriers, are especially preferred.

Other carriers that may be incorporated into drug layer 40, and/or drug layer 30, include carbohydrates that exhibit sufficient osmotic activity to be used alone or with other osmagents. Such carbohydrates comprise monosaccharides, disaccharides and polysaccharides. Representative examples include maltodextrins (i.e., glucose polymers produced by the hydrolysis of corn starch) and the sugars comprising lactose, glucose, raffinose, sucrose, mannitol, sorbitol, and the like. Preferred maltodextrins are those having a dextrose equivalence (DE) of 20 or less, preferably with a DE ranging from about 4 to about 20, and often 9-20. Maltodextrin having a DE of 9-12 has been found to be useful.

Drug layer 40 and drug layer 30 typically will be a substantially dry, <1% water by weight, composition formed by compression of the carrier, the benzimidazole derivative, and other excipients as one layer.

Drug layer 40 may be formed from particles by comminution that produces the size of the drug and the size of the accompanying polymer used in the fabrication of the drug layer, typically as a core containing the compound, according to the mode and the manner of the invention. The means for producing particles include granulation, extrusion, spray drying, sieving, lyophilization, crushing, grinding, jet milling, micronizing and chopping to produce the intended micron particle size. The process can be performed by size reduction equipment, such as a micropulverizer mill, a fluid energy grinding mill, a grinding mill, a roller compaction mill, a hammer mill, an attrition mill, a chaser mill, a ball mill, a vibrating ball mill, an impact pulverizer mill, a centrifugal pulverizer, a coarse crusher and a fine crusher. The size of the particle can be ascertained by screening, including a grizzly screen, a flat screen, a vibrating screen, a revolving screen, a shaking screen, an oscillating screen and a reciprocating screen. The processes and equipment for preparing drug and carrier particles are disclosed in Pharmaceutical Sciences, Remington, 17th Ed., pp. 1585-1594 (1985); Chemical Engineers Handbook, Perry, 6th Ed., pp. 21-13 to 21-19 (1984); Journal of Pharmaceutical Sciences, Parrot, Vol. 61, No. 6, pp. 813-829 (1974); and Chemical Engineer, Hixon, pp. 94-103 (1990).

First drug layer 30 comprises active agent in an admixture with selected excipients adapted to provide an osmotic activity gradient for driving fluid from an external environment through membrane 20 and for forming a deliverable drug formulation upon imbibition of fluid. The excipients may include a suitable suspending agent, also referred to herein as a drug carrier, and an osmotically active agent, i.e., an “osmagent,” such as salt. Other excipients such as lubricants, binders, etc. may also be included. It has been surprisingly found that when first component drug layer 30 comprises an osmotically active component, and a lower amount of active drug than in second component drug layer 40, an improved ascending rate of release can be created that provides a longer duration of ascending rate.

The osmotically active component in the first drug layer typically comprises an osmagent and one or more osmopolymer(s) having relatively small molecular weights which exhibit swelling as fluid is imbibed such that release of these osmopolymers through exit 60 occurs similar to that of drug layer 40.

The ratio of benzimidazole derivative concentration between the first drug layer and the second drug layer alters the release rate profile. Release rate profile is calculated as the difference between the maximum release rate and the release rate achieved at the first time point after start-up (for example, at 6 hours), divided by the average release rate between the two data points.

Drug layer 30 and drug layer 40 may optionally contain surfactants and disintegrants in both drug layers. Exemplary of the surfactants are those having an HLB value of about 3-25, such as polyethylene glycol 400 monostearate, glyceryl monostearate, polyoxyethylene-4-sorbitan monolaurate, polyoxyethylene-20-sorbitan monooleate, polyoxyethylene-20-sorbitan monopalmitate, polyoxyethylene-20-monolaurate, polyoxyethylene-40-stearate, sodium oleate and the like.

Disintegrants may be selected from starches, clays, celluloses, algins and gums and crosslinked starches, celluloses and polymers. Representative disintegrants include corn starch, potato starch, croscarmelose, crospovidone, sodium starch glycolate, Veegum HV, methylcellulose, agar, bentonite, carboxymethylcellulose, alginic acid, guar gum and the like.

Membrane 20 is formed to be permeable to the passage of an external fluid, such as water and biological fluids, and is substantially impermeable to the passage of benzimidazole derivative, osmagent, osmopolymer and the like. As such, it is semipermeable. The selectively semipermeable compositions used for forming membrane 20 are essentially nonerodible and substantially insoluble in biological fluids during the life of the dosage form.

Representative polymers for forming membrane 20 comprise semipermeable homopolymers, semipermeable copolymers, and the like. In one presently preferred embodiment, the compositions can comprise cellulose esters, cellulose ethers, and cellulose ester-ethers. The cellulosic polymers typically have a degree of substitution, “D.S.”, on their anhydroglucose unit from greater than 0 up to 3 inclusive. By degree of substitution is meant the average number of hydroxyl groups originally present on the anhydroglucose unit that are replaced by a substituting group, or converted into another group. The anhydroglucose unit can be partially or completely substituted with groups such as acyl, alkanoyl, alkenoyl, aroyl, alkyl, alkoxy, halogen, carboalkyl, alkylcarbamate, alkylcarbonate, alkylsulfonate, alkylsulfamate, semipermeable polymer forming groups, and the like. The semipermeable compositions typically include a member selected from the group consisting of cellulose acylate, cellulose diacylate, cellulose triacylate, cellulose triacetate, cellulose acetate, cellulose diacetate, cellulose triacetate, mono-, di- and tri-cellulose alkanylates, mono-, di-, and tri-alkenylates, mono-, di-, and tri-aroylates, and the like.

Exemplary polymers can include, for example, cellulose acetate have a D.S. of 1.8 to 2.3 and an acetyl content of 32 to 39.9%; cellulose diacetate having a D.S. of 1 to 2 and an acetyl content of 21 to 35%, cellulose triacetate having a D.S. of 2 to 3 and an acetyl content of 34 to 44.8%, and the like. More specific cellulosic polymers include cellulose propionate having a D.S. of 1.8 and a propionyl content of 38.5%; cellulose acetate propionate having an acetyl content of 1.5 to 7% and an acetyl content of 39 to 42%; cellulose acetate propionate having an acetyl content of 2.5 to 3%, an average propionyl content of 39.2 to 45%, and a hydroxyl content of 2.8 to 5.4%; cellulose acetate butyrate having a D.S. of 1.8, an acetyl content of 13 to 15%, and a butyryl content of 34 to 39%; cellulose acetate butyrate having an acetyl content of 2 to 29%, a butyryl content of 17 to 53%, and a hydroxyl content of 0.5 to 4.7%; cellulose triacylates having a D.S. of 2.6 to 3 such as cellulose trivalerate, cellulose trilamate, cellulose tripalmitate, cellulose trioctanoate, and cellulose tripropionate; cellulose diesters having a D.S. of 2.2 to 2.6 such as cellulose disuccinate, cellulose dipalmitate, cellulose dioctanoate, cellulose dicarpylate, and the like; mixed cellulose esters such as cellulose acetate valerate, cellulose acetate succinate, cellulose propionate succinate, cellulose acetate octanoate, cellulose valerate palmitate, cellulose acetate heptonate, and the like. Semipermeable polymers are known in U.S. Pat. No. 4,077,407 and they can be synthesized by procedures described in Encyclopedia of Polymer Science and Technology, Vol. 3, pages 325 to 354, 1964, published by Interscience Publishers, Inc., New York.

Additional semipermeable polymers for forming the semipermeable membrane can comprise, for example, cellulose acetaldehyde dimethyl acetate; cellulose acetate ethylcarbamate; cellulose acetate methylcarbamate; cellulose dimethylaminoacetate; semipermeable polyamide; semipermeable polyurethanes; semipermeable sulfonated polystyrenes; cross-linked selectively semipermeable polymers formed by the coprecipitation of a polyanion and a polycation as disclosed in U.S. Pat. Nos. 3,173,876; 3,276,586; 3,541,005; 3,541,006; and 3,546,142; semipermeable polymers as disclosed in U.S. Pat. No. 3,133,132; semipermeable polystyrene derivatives; semipermeable poly (sodium styrenesulfonate); semipermeable poly(vinylbenzyltremethylammonium chloride); semipermeable polymers, exhibiting a fluid permeability of 10-5 to 10-2 (cc. mil/cm hr.atm) expressed as per atmosphere of hydrostatic or osmotic pressure differences across a semipermeable membrane. The polymers are known to the art in U.S. Pat. Nos. 3,845,770; 3,916,899; and 4,160,020; and in Handbook of Common Polymers, by Scott, J. R., and Roff, W. J., 1971, published by CRC Press, Cleveland. Ohio.

Membrane 20 may also comprise a flux-regulating agent. The flux regulating agent is a compound added to assist in regulating the fluid permeability or flux through the membrane 20. The flux regulating agent can be a flux enhancing agent or a decreasing agent. The agent can be preselected to increase or decrease the liquid flux. Agents that produce a marked increase in permeability to fluids such as water are often essentially hydrophilic, while those that produce a marked decrease to fluids such as water are essentially hydrophobic. The amount of regulator in membrane 20 when incorporated therein generally is from about 0.01% to 20% by weight or more. The flux regulator agents in one embodiment that increase flux include, for example, polyhydric alcohols, polyalkylene glycols, polyalkylenediols, polyesters of alkylene glycols, and the like. Typical flux enhancers include polyethylene glycol 300, 400, 600, 1500, 4000, 6000, poly(ethylene glycol-co-propylene glycol), and the like; low molecular weight gylcols such as polypropylene glycol, polybutylene glycol and polyamylene glycol: the polyalkylenediols such as poly(1,3-propanediol), poly(1,4-butanediol), poly(1,6-hexanediol), and the like; aliphatic diols such as 1,3-butylene glycol, 1,4-pentamethylene glycol, 1,4-hexamethylene glycol, and the like; alkylene triols such as glycerine, 1,2,3-butanetriol, 1,2,4-hexanetriol, 1,3,6-hexanetriol and the like; esters such as ethylene glycol dipropionate, ethylene glycol butyrate, butylene glucol dipropionate, glycerol acetate esters, and the like. Representative flux decreasing agents include, for example, phthalates substituted with an alkyl or alkoxy or with both an alkyl and alkoxy group such as diethyl phthalate, dimethoxyethyl phthalate, dimethyl phthalate, and [di(2-ethylhexyl)phthalate], aryl phthalates such as triphenyl phthalate, and butyl benzyl phthalate; insoluble salts such as calcium sulphate, barium sulphate, calcium phosphate, and the like; insoluble oxides such as titanium oxide; polymers in powder, granule and like form such as polystyrene, polymethylmethacrylate, polycarbonate, and polysulfone; esters such as citric acid esters esterified with long chain alkyl groups; inert and substantially water impermeable fillers; resins compatible with cellulose based membrane forming materials, and the like.

Other materials that can be used to form membrane 20 for imparting flexibility and elongation properties to the wall, for making the membraneless-to-nonbrittle and to render tear strength, include, for example, phthalate plasticizers such as dibenzyl phthalate, dihexyl phthalate, butyl octyl phthalate, straight chain phthalates of six to eleven carbons, di-isononyl phthalte, di-isodecyl phthalate, and the like. The plasticizers include nonphthalates such as triacetin, dioctyl azelate, epoxidized tallate, tri-isoctyl trimellitate, tri-isononyl trimellitate, sucrose acetate isobutyrate, epoxidized soybean oil, and the like. The amount of plasticizer in a membrane when incorporated therein is about 0.01% to 20% weight, or higher.

Push layer 50 comprises an expandable layer in contacting layered arrangement with the second drug layer 40 as illustrated in FIG. 2. Push layer 50 comprises a polymer that imbibes an aqueous or biological fluid and swells to push the drug composition through the exit of the device.

The expandable layer comprises in one embodiment a hydroactivated composition that swells in the presence of water, such as that present in gastric fluids. Conveniently, it can comprise an osmotic composition comprising an osmotic solute that exhibits an osmotic pressure gradient across the semipermeable membrane against an external fluid present in the environment of use. In another embodiment, the hydro-activated layer comprises a hydrogel that imbibes and/or absorbs fluid into the layer through the outer semipermeable membrane. The semipermeable membrane is non-toxic. It maintains its physical and chemical integrity during operation and it is essentially free of interaction with the expandable layer.

The expandable layer in one preferred embodiment comprises a hydroactive layer comprising a hydrophilic polymer, also known as osmopolymers. The osmopolymers exhibit fluid imbibition properties. The osmopolymers are swellable, hydrophilic polymers, which osmopolymers interact with water and biological aqueous fluids and swell or expand to an equilibrium state. The osmopolymers exhibit the ability to swell in water and biological fluids and retain a significant portion of the imbibed fluid within the polymer structure. The osmopolymers swell or expand to a very high degree, usually exhibiting a 2 to 50 fold volume increase. The osmopolymers can be non-cross-linked or cross-linked. The swellable, hydrophilic polymers are in one embodiment lightly cross-linked, such cross-links being formed by covalent or ionic bonds or residue crystalline regions after swelling. The osmopolymers can be of plant, animal or synthetic origin.

The osmopolymers are hydrophilic polymers. Hydrophilic polymers suitable for the present purpose include poly(hydroxy-alkyl methacrylate) having a molecular weight of from 30,000 to 5,000,000; poly(vinylpyrrolidone) having a molecular weight of from 10,000 to 360,000; anionic and cationic hydrogels; polyelectrolytes complexes; poly(vinyl alcohol) having a low acetate residual, cross-linked with glyoxal, formaldehyde, or glutaraldehyde and having a degree of polymerization of from 200 to 30,000; a mixture of methyl cellulose, cross-linked agar and carboxymethyl cellulose; a mixture of hydroxypropyl methylcellulose and sodium carboxymethylcellulose; a mixture of hydroxypropyl ethylcellulose and sodium carboxymethyl cellulose, a mixture of sodium carboxymethylcellulose and methylcellulose, sodium carboxymethylcellulose; potassium carboxymethylcellulose; a water insoluble, water swellable copolymer formed from a dispersion of finely divided copolymer of maleic anhydride with styrene, ethylene, propylene, butylene or isobutylene crosslinked with from 0.001 to about 0.5 moles of saturated cross-linking agent per mole of maleic anhydride per copolymer; water swellable polymers of N-vinyl lactams; polyoxyethylene-polyoxypropylene gel; carob gum; polyacrylic gel; polyester gel; polyuria gel; polyether gel, polyamide gel; polycellulosic gel; polygum gel; initially dry hydrogels that imbibe and absorb water which penetrates the glassy hydrogel and lowers its glass temperature; and the like.

Representative of other osmopolymers are polymers that form hydrogels such as Carbopol™, acidic carboxypolymer, a polymer of acrylic acid cross-linked with a polyallyl sucrose, also known as carboxypolymethylene, and carboxyvinyl polymer having a molecular weight of 250,000 to 4,000,000; Cyanamer™ polyacrylamides; cross-linked water swellable indenemaleic anhydride polymers; Good-rite™ polyacrylic acid having a molecular weight of 80,000 to 200,000; Polyox™ polyethylene oxide polymer having a molecular weight of 100,000 to 5,000,000 and higher; starch graft copolymers; Aqua-Keeps™ acrylate polymer polysaccharides composed of condensed glucose units such as diester cross-linked polygluran; and the like. Representative polymers that form hydrogels are known to the prior art in U.S. Pat. No. 3,865,108; U.S. Pat. No. 4,002,173; U.S. Pat. No. 4,207,893; and in Handbook of Common Polymers, by Scott and Roff, published by the Chemical Rubber Co., Cleveland, Ohio. The amount of osmopolymer comprising a hydro-activated layer can be from about 5% to 100%.

The expandable layer in another manufacture can comprise an osmotically effective compound that comprises inorganic and organic compounds that exhibit an osmotic pressure gradient across a semipermeable membrane against an external fluid. The osmotically effective compounds, as with the osmopolymers, imbibe fluid into the osmotic system, thereby making available fluid to push against the inner wall, i.e., in some embodiments, the barrier layer and/or the membrane of the soft or hard capsule for pushing active agent from the dosage form. The osmotically effective compounds are known also as osmotically effective solutes, and also as osmagents. Osmotically effective solutes that can be used comprise magnesium sulfate, magnesium chloride, potassium sulfate, sodium sulfate, lithium sulfate, potassium acid phosphate, mannitol, urea, inositol, magnesium succinate, tartaric acid, carbohydrates such as raffinose, sucrose, glucose, lactose, sorbitol, and mixtures therefor. The amount of osmagent in can be from about 5% to 100% of the weight of the layer. The expandable layer optionally comprises an osmopolymer and an osmagent with the total amount of osmopolymer and osmagent equal to 100%. Osmotically effective solutes are known to the prior art as described in U.S. Pat. No. 4,783,337.

Protective subcoat, inner wall 90, is permeable to the passage of fluid entering the compartment defined by membrane 20. Wall 90 provides a lubricating function that facilitates the movement of first drug layer 30, second drug layer 40 and push layer 50 toward exit 60. Wall 90 may be formed from hydrophilic materials and excipients. Wall 90 promotes release of the drug composition from the compartment and reduces the amount of residual drug composition remaining in the compartment at the end of the delivery period, particularly when the slurry, suspension or solution of the drug composition that is being dispensed is highly viscous during the period of time in which it is being dispensed. In dosage forms with hydrophobic agents and no inner wall, it has been observed that significant residual amounts of drug may remain in the device after the period of delivery has been completed. In some instances, amounts of 20% or greater may remain in the dosage form at the end of a twenty-four hour period when tested in a release rate assay. Particularly in the case of active compounds having a high cost, such an improvement presents substantial economic advantages since it is not necessary to load the drug layer with an excess of drug to insure that the minimum amount of drug required will be delivered. Inner membrane 90 may be formed as a coating applied over the compressed core.

Wall 90 typically may be 0.01 to 5 mm thick, more typically 0.5 to 5 mm thick, and it comprises a member selected from hydrogels, gelatin, low molecular weight polyethylene oxides, e.g., less than 100,000 MW, hydroxyalkylcelluloses, e.g., hydroxyethylcellulose, hydroxypropylcellulose, hydroxyisopropylcelluose, hydroxybutylcellulose and hydroxyphenylcellulose, and hydroxyalkyl alkylcelluloses, e.g., hydroxypropyl methylcellulose, and mixtures thereof. The hydroxyalkylcelluloses comprise polymers having a 9,500 to 1,250,000 number-average molecular weight. For example, hydroxypropyl celluloses having number average molecular weights of 80,000 to 850,000 are useful. The wall 90 may be prepared from conventional solutions or suspensions of the aforementioned materials in aqueous solvents or inert organic solvents.

Preferred materials for the wall 90 include hydroxypropyl cellulose, hydroxyethyl cellulose, hydroxypropyl methyl cellulose, povidone [poly(vinylpyrrolidone)], polyethylene glycol, and mixtures thereof.

Most preferred are mixtures of hydroxypropyl cellulose and povidone, prepared in organic solvents, particularly organic polar solvents such as lower alkanols having 1-8 carbon atoms, preferably ethanol, mixtures of hydroxyethyl cellulose and hydroxypropyl methyl cellulose prepared in aqueous solution, and mixtures of hydroxyethyl cellulose and polyethylene glycol prepared in aqueous solution. Most preferably, the wall 90 comprises a mixture of hydroxypropyl cellulose and providone prepared in ethanol.

It is preferred that wall 90 comprises between about 50% and about 90% hydroxypropylcellulose identified as EF having an average molecular weight of about 80,000 and between about 10% and about 50% polyvinyl pyrrolidone identified as K29-32.

Conveniently, the weight of the wall 90 applied to the compressed core may be correlated with the thickness of the wall 90 and residual drug remaining in a dosage form in a release rate assay such as described herein. As such, during manufacturing operations, the thickness of the wall 90 may be controlled by controlling the weight of the wall 90 taken up in the coating operation.

When wall 90 is formed as a subcoat, i.e., by coating onto the tabletted composite including one or all of the first drug layer, second drug layer and push layer, the wall 90 can fill in surface irregularities formed on the core by the tabletting process. The resulting smooth external surface facilitates slippage between the coated composite core and the semipermeable membrane during dispensing of the drug, resulting in a lower amount of residual drug composition remaining in the device at the end of the dosing period. When wall 90 is fabricated of a gel-forming material, contact with water in the environment of use facilitates formation of the gel or gel-like inner coat having a viscosity that may promote and enhance slippage between membrane 20 and drug layer 30 and drug layer 40.

Pan coating may be conveniently used to provide the completed dosage form, except for the exit orifice. In the pan coating system, the wall-forming composition for the wall or the membrane, as the case may be, is deposited by successive spraying of the appropriate membrane composition onto the compressed trilayered or multilayered core comprising the drug layers, optional barrier layer and push layer, accompanied by tumbling in a rotating pan. A pan coater is used because of its availability at commercial scale. Other techniques can be used for coating the compressed core. Once coated, the membrane is dried in a forced-air oven or in a temperature and humidity controlled oven to free the dosage form of solvent(s) used in the manufacturing. Drying conditions will be conventionally chosen on the basis of available equipment, ambient conditions, solvents, coatings, coating thickness, and the like.

Other coating techniques can also be employed. For example, the membrane or walls of the dosage form may be formed in one technique using the air-suspension procedure. This procedure consists of suspending and tumbling the compressed core in a current of air and the semipermeable membrane forming composition, until the membrane is applied to the core. The air-suspension procedure is well suited for independently forming the membrane of the dosage form. The air-suspension procedure is described in U.S. Pat. No. 2,799,241; in J. Am. Pharm. Assoc., Vol. 48, pp. 451-459 (1959); and, ibid., Vol. 49, pp. 82-84 (1960). The dosage form also can be coated with a Wurster® air-suspension coater using, for example, methylene dichloride methanol as a cosolvent for the membrane forming material. An Aeromatic® air-suspension coater can be used employing a cosolvent.

In an embodiment, the sustained release dosage form of the invention is provided with at least one exit 60 as shown in FIG. 2. Exit 60 cooperates with the compressed core for the uniform release of drug from the dosage form. The exit can be provided during the manufacture of the dosage form or during drug delivery by the dosage form in a fluid environment of use.

One or more exit orifices are drilled in the drug layer end of the dosage form, and optional water soluble overcoats, which may be colored (e.g., Opadry colored coatings) or clear (e.g., Opadry Clear), may be coated on the dosage form to provide the finished dosage form.

Exit 60 may include an orifice that is formed or formable from a substance or polymer that erodes, dissolves or is leached from the outer membrane to thereby form an exit orifice. The substance or polymer may include, for example, an erodible poly(glycolic) acid or poly(lactic) acid in the semipermeable wall; a gelatinous filament; a water-removable poly(vinyl alcohol); a leachable compound, such as a fluid removable pore-former selected from the group consisting of inorganic and organic salt, oxide and carbohydrate.

An exit, or a plurality of exits, can be formed by leaching a member selected from the group consisting of sorbitol, lactose, fructose, glucose, mannose, galactose, talose, sodium chloride, potassium chloride, sodium citrate and mannitol to provide a uniform-release dimensioned pore-exit orifice.

The exit can have any shape, such as round, triangular, square, elliptical and the like for the uniform metered dose release of a drug from the dosage form.

The sustained release dosage form can be constructed with one or more exits in spaced-apart relation or one or more surfaces of the sustained release dosage form.

Drilling, including mechanical and laser drilling, through the semipermeable membrane can be used to form the exit orifice. Such exits and equipment for forming such exits are disclosed in U.S. Pat. No. 3,916,899, by Theeuwes and Higuchi and in U.S. Pat. No. 4,088,864, by Theeuwes, et al. It is presently preferred to utilize two exits of equal diameter. In a preferred embodiment, exit 60 penetrates through subcoat 90, if present, to drug layer 30.

Dosage forms in accordance with the embodiments depicted in FIG. 1 are manufactured by standard techniques. For example, the dosage form may be manufactured by the wet granulation technique. In the wet granulation technique, the drug and carrier are blended using an organic solvent, such as denatured anhydrous ethanol, as the granulation fluid. The remaining ingredients can be dissolved in a portion of the granulation fluid, such as the solvent described above, and this latter prepared wet blend is slowly added to the drug blend with continual mixing in the blender. The granulating fluid is added until a wet blend is produced, which wet mass blend is then forced through a predetermined screen onto oven trays. The blend is dried for 18 to 24 hours at 24° C. to 35° C. in a forced-air oven with or without controlled relative humidity. The dried granules are then sized. Next, magnesium stearate, or another suitable lubricant, is added to the drug granulation, and the granulation is put into milling jars and mixed on a jar mill for 10 minutes. The composition is pressed into a layer, for example, in a Manesty® press or a Korsch LCT press. For a trilayered core, granules or powders of the drug layer compositions and push layer composition are sequentially placed in an appropriately-sized die with intermediate compression steps being applied to each of the first two layers, followed by a final compression step after the last layer is added to the die to form the trilayered core. The intermediate compression typically takes place under a force of about 50-100 newtons. Final stage compression typically takes place at a force of 3500 newtons or greater, often 3500-5000 newtons. The compressed cores are fed to a dry coater press, e.g., Kilian® Dry Coater press, and subsequently coated with the membranematerials as described above.

In another embodiment, the drug and other ingredients comprising the drug layer are blended and pressed into a solid layer. The layer possesses dimensions that correspond to the internal dimensions of the area the layer is to occupy in the dosage form, and it also possesses dimensions corresponding to the push layer, if included, for forming a contacting arrangement therewith. The drug and other ingredients can also be blended with a solvent and mixed into a solid or semisolid form by conventional methods, such as ballmilling, calendering, stirring or rollmilling, and then pressed into a preselected shape. Next, if included, a layer of osmopolymer composition is placed in contact with the layer of drug in a like manner. The layering of the drug formulation and the osmopolymer layer can be fabricated by conventional two-layer press techniques. An analogous procedure may be followed for the preparation of the trilayered core. The compressed cores then may be coated with the wall material and the semipermeable membrane material as described above.

Another manufacturing process that can be used comprises blending the powdered ingredients for each layer in a fluid bed granulator. After the powdered ingredients are dry blended in the granulator, a granulating fluid, for example, poly(vinylpyrrolidone) in water or alcohol, is sprayed onto the powders. The coated powders are then dried in the granulator. This process granulates all the ingredients present therein while adding the granulating fluid. After the granules are dried, a lubricant, such as stearic acid or magnesium stearate, is mixed into the granulation using a blender e.g., V-blender or tote blender. The granules are then pressed in the manner described above.

Exemplary solvents suitable for manufacturing the dosage form components comprise aqueous or inert organic solvents that do not adversely harm the materials used in the system. The solvents broadly include members selected from the group consisting of aqueous solvents, alcohols, ketones, esters, ethers, aliphatic hydrocarbons, halogenated solvents, cycloaliphatics, aromatics, heterocyclic solvents and mixtures thereof. Typical solvents include acetone, diacetone alcohol, methanol, ethanol, isopropyl alcohol, butyl alcohol, methyl acetate, ethyl acetate, isopropyl acetate, n-butyl acetate, methyl isobutyl ketone, methyl propyl ketone, n-hexane, n-heptane, ethylene glycol monoethyl ether, ethylene glycol monoethyl acetate, methylene dichloride, ethylene dichloride, propylene dichloride, carbon tetrachloride nitroethane, nitropropane tetrachloroethane, ethyl ether, isopropyl ether, cyclohexane, cyclooctane, benzene, toluene, naphtha, 1,4-dioxane, tetrahydrofuran, diglyme, water, aqueous solvents containing inorganic salts such as sodium chloride, calcium chloride, and the like, and mixtures thereof such as acetone and water, acetone and methanol, acetone and ethyl alcohol, methylene dichloride and methanol, and ethylene dichloride and methanol.

One important consideration in the practice of this invention is the physical state of the benzimidazole derivative to be delivered by the dosage form. In certain embodiments, the benzimidazole derivatives may be in a paste, semi-solid, or liquid state. In such cases solid dosage forms may not be suitable for use in the practice of this invention. Instead, dosage forms capable of delivering substances in a paste, semi-solid, or liquid state should be used.

The present invention provides a liquid formulation of substances for use with oral osmotic devices. Oral osmotic devices for delivering liquid formulations and methods of using them are known in the art, for example, as described and claimed in the following U.S. patents owned by ALZA corporation: U.S. Pat. Nos. 6,419,952; 6,174,547; 6,551,613; 5,324,280; 4,111,201; and 6,174,547. Methods of using oral osmotic devices for delivering therapeutic agents at an ascending rate of release can be found in International Application Numbers WO 98/06380, WO 98/23263, and WO 99/62496. Should a liquid formulation approach be selected, care should be taken to reduce exposure of benzimidazole derivatives to water, particularly as part of the formulation, as water may cause the benzimidazole derivative(s) to degrade.

Exemplary liquid carriers for the present invention include lipophilic solvents (e.g., oils and lipids), surfactants, and hydrophilic solvents. Exemplary lipophilic solvents, for example, include, but are not limited to, Capmul PG-8, Caprol MPGO, Capryol 90, Plurol Oleique CC 497, Capmul MCM, Labrafac PG, N-Decyl Alcohol, Caprol 10G100, Oleic Acid, Vitamin E, Maisine 35-1, Gelucire 33/01, Gelucire 44/14, Lauryl Alcohol, Captex 355EP, Captex 500, Capylic/Caplic Triglyceride, Peceol, Caprol ET, Labrafil M2125 CS, Labrafac CC, Labrafil M 1944 CS, Captex 8277, Myvacet 9-45, Isopropyl Nyristate, Caprol PGE 860, Olive Oil, Plurol Oleique, Peanut Oil, Captex 300 Low C6, and Capric Acid.

Exemplary surfactants for example, include, but are not limited to, Vitamin E TPGS, Cremophor (grades EL, EL-P, and RH40), Labrasol, Tween (grades 20, 60, 80), Pluronic (grades L-31, L-35, L-42, L-64, and L-121), Acconon S-35, Solutol HS-15, and Span (grades 20, and 80). Exemplary hydrophilic solvents for example, include, but are not limited to, Isosorbide Dimethyl Ether, Polyethylene Glycol (PEG grades 300, 400, 600, 3000, 4000, 6000, and 8000) and Propylene Glycol (PG).

The skilled practitioner will understand that any formulation comprising a sufficient dosage of benzimidazole derivative solubilized in a liquid carrier suitable for administration to a subject and for use in an osmotic device can be used in the present invention. In one exemplary embodiment of the present invention, the liquid carrier is PG, Solutol, Cremophor EL, or a combination thereof.

The liquid formulation according to the present invention can also comprise, for example, additional excipients such as an antioxidant, permeation enhancer and the like. Antioxidants can be provided to slow or effectively stop the rate of any autoxidizable material present in the capsule. Representative antioxidants can comprise a member selected from the group of ascorbic acid; alpha tocopherol; ascorbyl palmitate; ascorbates; isoascorbates; butylated hydroxyanisole; butylated hydroxytoluene; nordihydroguiaretic acid; esters of garlic acid comprising at least 3 carbon atoms comprising a member selected from the group consisting of propyl gallate, octyl gallate, decyl gallate, decyl gallate; 6-ethoxy-2,2,4-trimethyl-1,2-dihydro-quinoline; N-acetyl-2,6-di-t-butyl-p-aminophenol; butyl tyrosine; 3-tertiarybutyl-4-hydroxyanisole; 2-tertiary-butyl-4-hydroxyanisole; 4-chloro-2,6-ditertiary butyl phenol; 2,6-ditertiary butyl p-methoxy phenol; 2,6-ditertiary butyl-p-cresol: polymeric antioxidants; trihydroxybutyro-phenone physiologically acceptable salts of ascorbic acid, erythorbic acid, and ascorbyl acetate; calcium ascorbate; sodium ascorbate; sodium bisulfite; and the like. The amount of antioxidant used for the present purposes, for example, can be about 0.001% to 25% of the total weight of the composition present in the lumen. Antioxidants are known to the prior art in U.S. Pat. Nos. 2,707,154; 3,573,936; 3,637,772; 4,038,434; 4,186,465 and 4,559,237, each of which is hereby incorporated by reference in its entirety for all purposes.

The inventive liquid formulation can comprise permeation enhancers that facilitate absorption of the drug in the environment of use. Such enhancers can, for example, open the so-called “tight junctions” in the gastrointestinal tract or modify the effect of cellular components, such a p-glycoprotein and the like. Suitable enhancers can include alkali metal salts of salicyclic acid, such as sodium salicylate, caprylic or capric acid, such as sodium caprylate or sodium caprate, and the like. Enhancers can include, for example, the bile salts, such as sodium deoxycholate. Various p-glycoprotein modulators are described in U.S. Pat. Nos. 5,112,817 and 5,643,909. Various other absorption enhancing compounds and materials are described in U.S. Pat. No. 5,824,638. Enhancers can be used either alone or as mixtures in combination with other enhancers.

In certain embodiments, the inventive substances are administered as a self-emulsifying formulation. Like the other liquid carriers, the surfactant functions to prevent aggregation, reduce interfacial tension between constituents, enhance the free-flow of constituents, and lessen the incidence of constituent retention in the dosage form. The emulsion formulation of this invention comprises a surfactant that imparts emulsification. Exemplary surfactants can also include, for example, in addition to the surfactants listed above, a member selected from the group consisting of polyoxyethylenated castor oil comprising ethylene oxide in the concentration of 9 to 15 moles, polyoxyethylenated sorbitan monopalmitate, mono and tristearate comprising 20 moles of ethylene oxide, polyoxyethylenated sorbitan monostearate comprising 4 moles of ethylene oxide, polyoxyethylenated sorbitan trioleate comprising 20 moles of ethylene oxide, polyoxyethylene lauryl ether, polyoxyethylenated stearic acid comprising 40 to 50 moles of ethylene oxide, polyoxyethylenated stearyl alcohol comprising 2 moles of ethylene oxide, and polyoxyethylenated oleyl alcohol comprising 2 moles of ethylene oxide. The surfactants may be available from Atlas Chemical Industries.

The emulsified drug formulations of the present invention can initially comprise an oil and a non-ionic surfactant. The oil phase of the emulsion comprises any pharmaceutically acceptable oil that is not immiscible with water. The oil can be an edible liquid such as a non-polar ester of an unsaturated fatty acid, derivatives of such esters, or mixtures of such esters. The oil can be vegetable, mineral, animal or marine in origin. Examples of non-toxic oils can also include, for example, in addition to the surfactants listed above, a member selected from the group consisting of peanut oil, cottonseed oil, sesame oil, corn oil, almond oil, mineral oil, castor oil, coconut oil, palm oil, cocoa butter, safflower, a mixture of mono- and diglycerides of 16 to 18 carbon atoms, unsaturated fatty acids, fractionated triglycerides derived from coconut oil, fractionated liquid triglycerides derived from short chain 10 to 15 carbon atoms fatty acids, acetylated monoglycerides, acetylated diglycerides, acetylated triglycerides, olein known also as glyceral trioleate, palmitin known as glyceryl tripalmitate, stearin known also as glyceryl tristearate, lauric acid hexylester, oleic acid oleylester, glycolyzed ethoxylated glycerides of natural oils, branched fatty acids with 13 molecules of ethyleneoxide, and oleic acid decylester. The concentration of oil, or oil derivative in the emulsion formulation can be from about 1 wt % to about 40 wt %, with the wt % of all constituents in the emulsion preparation equal to 100 wt %. The oils are disclosed in Pharmaceutical Sciences by Remington, 17th Ed., pp. 403-405, (1985) published by Mark Publishing Co., in Encyclopedia of Chemistry, by Van Nostrand Reinhold, 4th Ed., pp. 644-645, (1984) published by Van Nostrand Reinhold Co.; and in U.S. Pat. No. 4,259,323.

The amount of benzimidazole derivative incorporated in the dosage forms of the present invention is generally from about 10% to about 90% by weight of the composition depending upon the therapeutic indication and the desired administration period, e.g., every 12 hours, every 24 hours, and the like. Depending on the dose of benzimidazole derivative desired to be administered, one or more of the dosage forms can be administered.

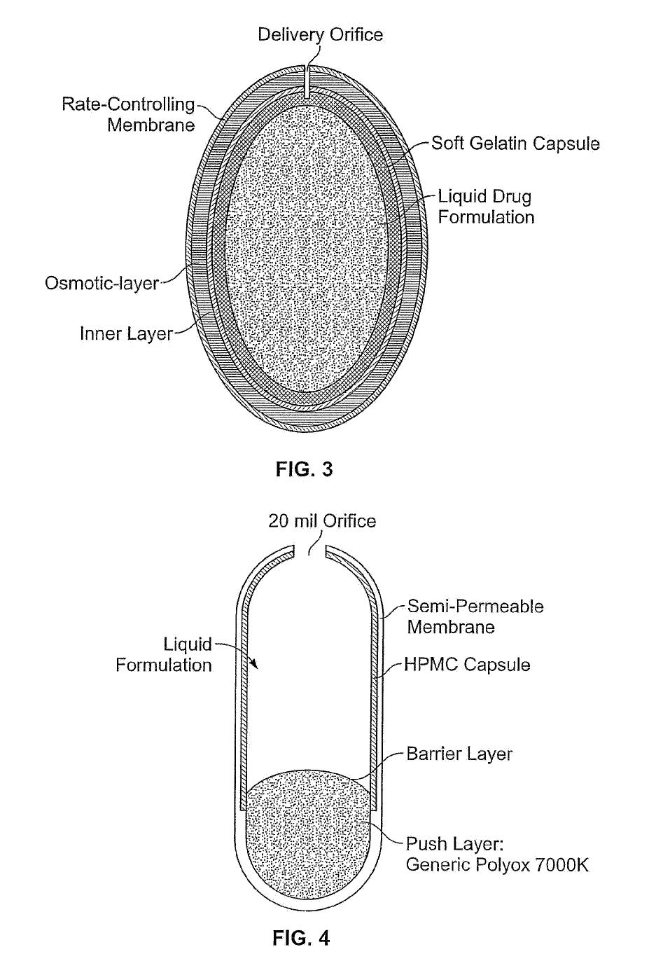

The osmotic dosage forms of the present invention can possess two distinct forms, a soft capsule form (shown in FIG. 3) and a hard capsule form (shown in FIG. 4). The soft capsule, as used by the present invention, preferably in its final form comprises one piece. The one-piece capsule is of a sealed construction encapsulating the drug formulation therein. The capsule can be made by various processes including the plate process, the rotary die process, the reciprocating die process, and the continuous process. An example of the plate process is as follows. The plate process uses a set of molds. A warm sheet of a prepared capsule lamina-forming material is laid over the lower mold and the formulation poured on it. A second sheet of the lamina-forming material is placed over the formulation followed by the top mold. The mold set is placed under a press and a pressure applied, with or without heat, to form a unit capsule. The capsules are washed with a solvent for removing excess agent formulation from the exterior of the capsule, and the air-dried capsule is encapsulated with a semipermeable wall. The rotary die process uses two continuous films of capsule lamina-forming material that are brought into convergence between a pair of revolving dies and an injector wedge. The process fills and seals the capsule in dual and coincident operations. In this process, the sheets of capsule lamina-forming material are fed over guide rolls, and then down between the wedge injector and the die rolls. The agent formulation to be encapsulated flows by gravity into a positive displacement pump. The pump meters the agent formulation through the wedge injector and into the sheets between the die rolls. The bottom of the wedge contains small orifices lined up with the die pockets of the die rolls. The capsule is about half-sealed when the pressure of pumped agent formulation forces the sheets into the die pockets, wherein the capsules are simultaneously filled, shaped, hermetically sealed and cut from the sheets of lamina-forming materials. The sealing of the capsule is achieved by mechanical pressure on the die rolls and by heating of the sheets of lamina-forming materials by the wedge. After manufacture, the agent formulation-filled capsules are dried in the presence of forced air, and a semipermeable lamina encapsulated thereto.

The reciprocating die process produces capsules by leading two films of capsule lamina-forming material between a set of vertical dies. The dies as they close, open, and close perform as a continuous vertical plate forming row after row of pockets across the film. The pockets are filled with an inventive formulation, and as the pockets move through the dies, they are sealed, shaped, and cut from the moving film as capsules filled with agent formulation. A semipermeable encapsulating lamina is coated thereon to yield the capsule. The continuous process is a manufacturing system that also uses rotary dies, with the added feature that the process can successfully fill active agent in dry powder form into a soft capsule, in addition to encapsulating liquids. The filled capsule of the continuous process is encapsulated with a semipermeable polymeric material to yield the capsule. Procedures for manufacturing soft capsules are disclosed in U.S. Pat. No. 4,627,850 and U.S. Pat. No. 6,419,952.

The dosage forms of the present invention can also be made from an injection-moldable composition by an injection-molding technique. Injection-moldable compositions provided for injection-molding into the semipermeable membrane comprise a thermoplastic polymer, or the compositions comprise a mixture of thermoplastic polymers and optional injection-molding ingredients. The thermoplastic polymer that can be used for the present purpose comprise polymers that have a low softening point, for example, below 200° C., preferably within the range of 40° C. to 180° C. The polymers, are preferably synthetic resins, addition polymerized resins, such as polyamides, resins obtained from diepoxides and primary alkanolamines, resins of glycerine and phthalic anhydrides, polymethane, polyvinyl resins, polymer resins with end-positions free or esterified carboxyl or caboxamide groups, for example with acrylic acid, acrylic amide, or acrylic acid esters, polycaprolactone, and its copolymers with dilactide, diglycolide, valerolactone and decalactone, a resin composition comprising polycaprolactone and polyalkylene oxide, and a resin composition comprising polycaprolactone, a polyalkylene oxide such as polyethylene oxide, poly(cellulose) such as poly(hydroxypropylmethylcellulose), poly(hydroxyethylmethylcellulose), and poly(hydroxypropylcellulose). The membrane forming composition can comprise optional membrane-forming ingredients such as polyethylene glycol, talcum, polyvinylalcohol, lactose, or polyvinyl pyrrolidone. The compositions for forming an injection-molding polymer composition can comprise 100% thermoplastic polymer. The composition in another embodiment comprises 10% to 99% of a thermoplastic polymer and 1% to 90% of a different polymer with the total equal to 100%. The invention provides also a thermoplastic polymer composition comprising 1% to 98% of a first thermoplastic polymer, 1% to 90% of a different, second polymer and 1% to 90% of a different, third polymer with all polymers equal to 100%. Representation composition comprises 20% to 90% of thermoplastic polycaprolactone and 10% to 80% of poly(alkylene oxide); a composition comprising 20% to 90% polycaprolactone and 10% to 60% of poly(ethylene oxide) with the ingredients equal to 100%; a composition comprising 10% to 97% of polycaprolactone, 10% to 97% poly(alkylene oxide), and 1% to 97% of poly(ethylene glycol) with all ingredients equal to 100%; a composition comprising 20% to 90% polycaprolactone and 10% to 80% of poly(hydroxypropylcellulose) with all ingredients equal to 100%; and a composition comprising 1% to 90% polycaprolactone, 1% to 90% poly(ethylene oxide), 1% to 90% poly(hydroxypropylcellulose) and 1% to 90% poly(ethylene glycol) with all ingredients equal to 100%. The percent expressed is weight percent wt %.

In another embodiment of the invention, a composition for injection-molding to provide a membrane can be prepared by blending a composition comprising a polycaprolactone 63 wt %, polyethylene oxide 27 wt %, and polyethylene glycol 10 wt % in a conventional mixing machine, such as a Moriyama™ Mixer at 65° C. to 95° C., with the ingredients added to the mixer in the following addition sequence, polycaprolactone, polyethylene oxide and polyethylene glycol. In one example, all the ingredients are mixed for 135 minutes at a rotor speed of 10 to 20 rpm. Next, the blend is fed to a Baker Perkins Kneader™ extruder at 80° C. to 90° C., at a pump speed of 10 rpm and a screw speed of 22 rpm, and then cooled to 10° C. to 12° C., to reach a uniform temperature. Then, the cooled extruded composition is fed to an Albe Pelletizer, converted into pellets at 250° C., and a length of 5 mm. The pellets next are fed into an injection-molding machine, an Arburg Allrounder™ at 200° F. to 350° C. (93° C. to 177° C.), heated to a molten polymeric composition, and the liquid polymer composition forced into a mold cavity at high pressure and speed until the mold is filled and the composition comprising the polymers are solidified into a preselected shape. The parameters for the injection-molding consists of a band temperature through zone 1 to zone 5 of the barrel of 195° F. (91° C.) to 375° F., (191° C.), an injection-molding pressure of 1818 bar, a speed of 55 cm3/s, and a mold temperature of 75° C. The injection-molding compositions and injection-molding procedures are disclosed in U.S. Pat. No. 5,614,578.

Alternatively, the capsule can be made conveniently in two parts, with one part (the “cap”) slipping over and capping the other part (the “body”) as long as the capsule is deformable under the forces exerted by the expandable layer and seals to prevent leakage of the liquid, active agent formulation from between the telescoping portions of the body and cap. The two parts completely surround and capsulate the internal lumen that contains the liquid, active agent formulation, which can contain useful additives. The two parts can be fitted together after the body is filled with a preselected formulation. The assembly can be done by slipping or telescoping the cap section over the body section, and sealing the cap and body, thereby completely surrounding and encapsulating the formulation of active agent.

Soft capsules typically have a wall thickness that is greater than the wall thickness of hard capsules. For example, soft capsules can, for example, have a wall thickness on the order of 10-40 mils, about 20 mils being typical, whereas hard capsules can, for example, have a wall thickness on the order of 2-6 mils, about 4 mils being typical.

In one embodiment of the dosage system, a soft capsule can be of single unit construction and can be surrounded by an unsymmetrical hydro-activated layer as the expandable layer. The expandable layer will generally be unsymmetrical and have a thicker portion remote from the exit orifice. As the hydro-activated layer imbibes and/or absorbs external fluid, it expands and applies a push pressure against the wall of the capsule and optional barrier layer and forces active agent formulation through the exit orifice. The presence of an unsymmetrical layer functions to assure that the maximum dose of agent is delivered from the dosage form, as the thicker section of layer distant from passageway swells and moves towards the orifice.

In yet another configuration, the expandable layer can be formed in discrete sections that do not entirely encompass an optionally barrier layer-coated capsule. The expandable layer can be a single element that is formed to fit the shape of the capsule at the area of contact. The expandable layer can be fabricated conveniently by tableting to form the concave surface that is complementary to the external surface of the barrier-coated capsule. Appropriate tooling such as a convex punch in a conventional tableting press can provide the necessary complementary shape for the expandable layer. In this case, the expandable layer is granulated and compressed, rather than formed as a coating. The methods of formation of an expandable layer by tableting are well known, having been described, for example in U.S. Pat. Nos. 4,915,949; 5,126,142; 5,660,861; 5,633,011; 5,190,765; 5,252,338; 5,620,705; 4,931,285; 5,006,346; 5,024,842; and 5,160,743.

In some embodiments, a barrier layer can be first coated onto the capsule and then the tableted, expandable layer is attached to the barrier-coated capsule with a biologically compatible adhesive. Suitable adhesives include, for example, starch paste, aqueous gelatin solution, aqueous gelatin/glycerin solution, acrylate-vinylacetate based adhesives such as Duro-Tak adhesives (National Starch and Chemical Company), aqueous solutions of water soluble hydrophilic polymers such as hydroxypropyl methyl cellulose, hydroxymethyl cellulose, hydroxyethyl cellulose, and the like. That intermediate dosage form can be then coated with a semipermeable layer. The exit orifice is formed in the side or end of the capsule opposite the expandable layer section. As the expandable layer imbibes fluid, it will swell. Since it is constrained by the semipermeable layer, as it expands it will compress the barrier-coated capsule and express the liquid, active agent formulation from the interior of the capsule into the environment of use.

The hard capsules are typically composed of two parts, a cap and a body, which are fitted together after the larger body is filled with a preselected appropriate formulation. This can be done by slipping or telescoping the cap section over the body section, thus completely surrounding and encapsulating the useful agent formulation. Hard capsules can be made, for example, by dipping stainless steel molds into a bath containing a solution of a capsule lamina-forming material to coat the mold with the material. Then, the molds are withdrawn, cooled, and dried in a current of air. The capsule is stripped from the mold and trimmed to yield a lamina member with an internal lumen. The engaging cap that telescopically caps the formulation receiving body is made in a similar manner. Then, the closed and filled capsule can be encapsulated with a semipermeable lamina. The semipermeable lamina can be applied to capsule parts before or after parts and are joined into the final capsule. In another embodiment, the hard capsules can be made with each part having matched locking rings near their opened end that permit joining and locking together the overlapping cap and body after filling with formulation. In this embodiment, a pair of matched locking rings are formed into the cap portion and the body portion, and these rings provide the locking means for securely holding together the capsule. The capsule can be manually filled with the formulation, or they can be machine filled with the formulation. In the final manufacture, the hard capsule is encapsulated with a semipermeable lamina permeable to the passage of fluid and substantially impermeable to the passage of useful agent. Methods of forming hard cap dosage forms are described in U.S. Pat. No. 6,174,547, U.S. Pat. Nos. 6,596,314, 6,419,952, and 6,174,547.

The hard and soft capsules can comprise, for example, gelatin; gelatin having a viscosity of 15 to 30 millipoises and a bloom strength up to 150 grams; gelatin having a bloom value of 160 to 250; a composition comprising gelatin, glycerine, water and titanium dioxide; a composition comprising gelatin, erythrosin, iron oxide and titanium dioxide; a composition comprising gelatin, glycerine, sorbitol, potassium sorbate and titanium dioxide; a composition comprising gelatin, acacia glycerine, and water; and the like. Materials useful for forming capsule membrane are known in U.S. Pat. Nos. 4,627,850; and in 4,663,148. Alternatively, the capsules can be made out of materials other than gelatin (see for example, products made by BioProgres plc).

The capsules typically can be provided, for example, in sizes from about 3 to about 22 minims (1 minim being equal to 0.0616 ml) and in shapes of oval, oblong or others. They can be provided in standard shape and various standard sizes, conventionally designated as (000), (00), (0), (1), (2), (3), (4), and (5). The largest number corresponds to the smallest size. Non-standard shapes can be used as well. In either case of soft capsule or hard capsule, non-conventional shapes and sizes can be provided if required for a particular application.

The osmotic devices of the present invention may comprise a semipermeable membrane permeable to the passage of exterior biological fluid and substantially impermeable to the passage of benzimidazole derivative formulation. The selectively permeable compositions used for forming the membrane are essentially non-erodible and they are insoluble in biological fluids during the life of the osmotic system. The semipermeable membrane comprises a composition that does not adversely affect the host, the benzimidazole derivative formulation, an osmopolymer, osmagent and the like. Materials useful in the formation of a semipermeable membrane are disclosed elsewhere herein.

The semipermeable membrane can also comprise a flux regulating agent. Materials useful flux regulating agents are disclosed elsewhere herein. Other materials that can be used to form the semipermeable membrane for imparting flexibility and elongation properties to the semipermeable membrane are also disclosed elsewhere herein.

The semipermeable membrane surrounds and forms a compartment containing a one or a plurality of layers, one of which is an expandable layer which in some embodiments, can contain osmotic agents. The composition of such expandable layers is disclosed elsewhere herein.

In certain solid and liquid embodiments, the dosage forms further can comprise a barrier layer. The barrier layer in certain embodiments is deformable under the pressure exerted by the expandable layer and will be impermeable (or less permeable) to fluids and materials that can be present in the expandable layer, the liquid active agent formulation and in the environment of use, during delivery of the active agent formulation. A certain degree of permeability of the barrier layer can be permitted if the delivery rate of the active agent formulation is not detrimentally affected. However, it is preferred that barrier layer not completely transport through it fluids and materials in the dosage form and the environment of use during the period of delivery of the active agent. The barrier layer can be deformable under forces applied by expandable layer so as to permit compression of capsule to force the liquid, active agent formulation from the exit orifice. In some embodiments, the barrier layer will be deformable to such an extent that it create a seal between the expandable layer and the semipermeable layer in the area where the exit orifice is formed. In that manner, the barrier layer will deform or flow to a limited extent to seal the initially, exposed areas of the expandable layer and the semipermeable layer when the exit orifice is being formed, such as by drilling or the like, or during the initial stages of operation. When sealed, the only avenue for liquid permeation into the expandable layer is through the semipermeable layer, and there is no back-flow of fluid into the expandable layer through the exit orifice.

Suitable materials for forming the barrier layer can include, for example, polyethylene, polystyrene, ethylene-vinyl acetate copolymers, polycaprolactone and Hytrel™ polyester elastomers (Du Pont), cellulose acetate, cellulose acetate pseudolatex (such as described in U.S. Pat. No. 5,024,842), cellulose acetate propionate, cellulose acetate butyrate, ethyl cellulose, ethyl cellulose pseudolatex (such as Surelease™ as supplied by Colorcon, West Point, Pa. or Aquacoat™ as supplied by FMC Corporation, Philadelphia, Pa.), nitrocellulose, polylactic acid, poly-glycolic acid, polylactide glycolide copolymers, collagen, polyvinyl alcohol, polyvinyl acetate, polyethylene vinylacetate, polyethylene teraphthalate, polybutadiene styrene, polyisobutylene, polyisobutylene isoprene copolymer, polyvinyl chloride, polyvinylidene chloride-vinyl chloride copolymer, copolymers of acrylic acid and methacrylic acid esters, copolymers of methylmethacrylate and ethylacrylate, latex of acrylate esters (such as Eudragit™ supplied by RohmPharma, Darmstaat, Germany), polypropylene, copolymers of propylene oxide and ethylene oxide, propylene oxide ethylene oxide block copolymers, ethylenevinyl alcohol copolymer, polysulfone, ethylene vinylalcohol copolymer, polyxylylenes, polyalkoxysilanes, polydimethyl siloxane, polyethylene glycol-silicone elastomers, electromagnetic irradiation crosslinked acrylics, silicones, or polyesters, thermally crosslinked acrylics, silicones, or polyesters, butadiene-styrene rubber, and blends of the above.

Preferred materials can include cellulose acetate, copolymers of acrylic acid and methacrylic acid esters, copolymers of methylmethacrylate and ethylacrylate, and latex of acrylate esters. Preferred copolymers can include poly (butyl methacrylate), (2-dimethylaminoethyl)methacrylate, methyl methacrylate) 1:2:1, 150,000, sold under the trademark EUDRAGIT E; poly(ethyl acrylate, methyl methacrylate) 2:1, 800,000, sold under the trademark EUDRAGIT NE 30 D; poly(methacrylic acid, methyl methacrylate) 1:1,135,000, sold under the trademark EUDRAGIT L; poly(methacrylic acid, ethyl acrylate) 1:1, 250,000, sold under the trademark EUDRAGIT L; poly(methacrylic acid, methyl methacrylate) 1:2,135,000, sold under the trademark EUDRAGIT S; poly(ethyl acrylate, methyl methacrylate, trimethylammonioethyl methacrylate chloride) 1:2:0.2, 150,000, sold under the trademark EUDRAGIT RL; poly(ethyl acrylate, methyl methacrylate, trimethylammonioethyl methacrylate chloride) 1:2:0.1, 150,000, sold as EUDRAGIT RS. In each case, the ratio x:y:z indicates the molar proportions of the monomer units and the last number is the number average molecular weight of the polymer. Especially preferred are cellulose acetate containing plasticizers such as acetyl tributyl citrate and ethylacrylate methylmethylacrylate copolymers such as Eudragit NE.

The foregoing materials for use as the barrier layer can be formulated with plasticizers to make the barrier layer suitably deformable such that the force exerted by the expandable layer will collapse the compartment formed by the barrier layer to dispense the liquid, active agent formulation. Examples of typical plasticizers are as follows: polyhydric alcohols, triacetin, polyethylene glycol, glycerol, propylene glycol, acetate esters, glycerol triacetate, triethyl citrate, acetyl triethyl citrate, glycerides, acetylated monoglycerides, oils, mineral oil, castor oil and the like. The plasticizers can be blended into the material in amounts of 10-50 weight percent based on the weight of the material.

The various layers forming the barrier layer, expandable layer and semipermeable layer can be applied by conventional coating methods such as described in U.S. Pat. No. 5,324,280. While the barrier layer, expandable layer and semipermeable membrane have been illustrated and described for convenience as single layers, each of those layers can be composites of several layers. For example, for particular applications it may be desirable to coat the capsule with a first layer of material that facilitates coating of a second layer having the permeability characteristics of the barrier layer. In that instance, the first and second layers comprise the barrier layer. Similar considerations would apply to the semipermeable layer and the expandable layer.

The exit orifice can be formed by mechanical drilling, laser drilling, eroding an erodible element, extracting, dissolving, bursting, or leaching a passageway former from the composite wall. The exit orifice can be a pore formed by leaching sorbitol, lactose or the like from a membrane or layer as disclosed in U.S. Pat. No. 4,200,098. This patent discloses pores of controlled-size porosity formed by dissolving, extracting, or leaching a material from a wall, such as sorbitol from cellulose acetate. A preferred form of laser drilling is the use of a pulsed laser that incrementally removes material from the composite membrane to the desired depth to form the exit orifice.

Additional potentially useful information may be found in WO 2004/103291, entitled “Extended Release Compositions of Proton Pump Inhibitor.”

FIGS. 3A-3C illustrate another exemplary dosage form, known in the art and described in U.S. Pat. Nos. 5,534,263; 5,667,804; and 6,020,000. Briefly, a cross-sectional view of a dosage form 80 is shown prior to ingestion into the gastrointestinal tract in FIG. 3A. The dosage form is comprised of a cylindrically shaped matrix 82 comprising an inventive substance. Ends 84, 86 of matrix 82 are preferably rounded and convex in shape in order to ensure ease of ingestion. Bands 88, 90, and 92 concentrically surround the cylindrical matrix and are formed of a material that is relatively insoluble in an aqueous environment. Suitable materials are set forth in the patents noted above and elsewhere herein.

After ingestion of dosage form 80, regions of matrix 82 between bands 88, 90, 92 begin to erode, as illustrated in FIG. 3B. Erosion of the matrix initiates release of the inventive substance into the fluidic environment of the G.I. tract. As the dosage form continues transit through the G.I. tract, the matrix continues to erode, as illustrated in FIG. 3C. Here, erosion of the matrix has progressed to such an extent that the dosage form breaks into three pieces, 94, 96, 98. Erosion will continue until the matrix portions of each of the pieces have completely eroded. Bands 94, 96, 98 will thereafter be expelled from the G.I. tract.

Other approaches to achieving sustained release of drugs from oral dosage forms are known in the art. For example, diffusion systems such as reservoir devices and matrix devices, dissolution systems such as encapsulated dissolution systems (including, for example, “tiny time pills”) and matrix dissolution systems, combination diffusion/dissolution systems and ion-exchange resin systems are known and are disclosed in Remington's Pharmaceutical Sciences, 1990 ed., pp. 1682-1685. Dosage forms that operate in accordance with these other approaches are encompassed by the scope of the disclosure herein to the extent that the drug release characteristics and/or the blood plasma concentration characteristics as recited herein and in the claims describe those dosage forms either literally or equivalently.

U.S. Pat. Nos. 5,871,778 and 5,656,299 disclose sustained microsphere formulations having almost zero order rate of release of active component when administered to a patient. U.S. Pat. Nos. 5,654,008; 5,650,173; 5,770,231; 6,077,843; 6,368,632; and 5,965,168 disclose sustained-release microparticle compositions and their use for controlled delivery of active agents.

Benzimidazole derivatives according to the invention may be unstable when introduced to an acid environment, such as the stomach. In certain embodiments, chemical stabilization of the benzimidazole derivatives according to the invention are provided. For instance, the stabilization techniques of U.S. Pat. No. 5,035,899 to Saeki et al., US. Published Patent Application 2004/0248939 to Sugaya et al., or of European Patent Application EP 1004305 to Ukai et al., may be used in the practice of this invention. In an embodiment, the inventive dosage forms comprise a chemical stabilizer. In preferable embodiments, the chemical stabilizer comprises sodium carbonate, potassium carbonate, sodium hydroxide, potassium hydroxide, aminoalkyl methaacrylate copolymer E, arginine aspartate, hydroxypropyl cellulose and/or crospovidone, meglumine, calcium salts and mixtures thereof.

In an embodiment, the chemical stabilizers comprise a metal oxide and/or a metal hydroxide. The metal oxide and metal hydroxide usable in the present invention are preferably those of which a 1% aqueous solution or a 1% aqueous suspension has a pH of 8.0 or more, and examples of the metal oxide include medical magnesium oxide, magnesium silicate (2MgO.3SiO₂.xH₂O), dry aluminum hydroxide gel (Al₂O₃. xH₂O), magnesium metasilicate aluminate (Al₂O₃.MgO₂SiO₂.x H₂O) and the like. Particularly, magnesium oxide can be suitably used.

Preferable magnesium oxides are those that are available for medical use and that have an excellent reactivity to acid and neutralization ability. As these magnesium oxides, magnesium oxide obtained by a usual production method and commercially available magnesium oxide can be used, and preferable is one obtained by calcination at low temperature, so-called, calcining magnesia. The magnesium oxide calcined at a temperature of about 500 to about 1000.degree. C. is generally preferable, and particularly from the viewpoint of neutralization ability the magnesium oxide calcined at a temperature of about 600 to about 900.degree. C. is preferable, and the magnesium oxide calcined at about 800.degree. C. is most preferable. Among these magnesium oxides, favorable is the one that neutralizes the environment prior to the release of the acid labile active ingredient by the disintegration of the preparation in stomach and has the function to enhance the remaining ratio of the active ingredient. Such magnesium oxide is preferably the one that has usually a BET specific surface area of about 10 m.sup.2/g to about 50 m.sup.2/g, preferably about 20 m.sup.2/g to about 5 m.sup.2/g.

In the present invention, a BET specific surface area means the specific surface area measured by nitrogen gas adsorption method, and the specific surface area containing the surface of given amount magnesium oxide and its cavity in which nitrogen gas can enter is determined by the amount of adsorbed nitrogen gas.

The magnesium oxide includes, for example, commercially available heavy magnesium oxide (manufactured by Kyowa Kagaku Kogyo K. K.), heavy magnesium oxide (Tomita Pharmaceutical Co. Ltd.), heavy N magnesium oxide (manufactured by Kyowa Kagaku Kogyo K. K.), light magnesium oxide (manufactured by Kyowa Kagaku Kogyo K. K.) and the like. Particularly heavy N magnesium oxide (manufactured by Kyowa Kagaku Kogyo K. K.) is preferable.

The metal hydroxide includes, for example, medical magnesium hydroxide, aluminum hydroxide, synthetic hydrotalcite (Mg₆Al₂OH)₁₆CO₂-4H₂O), co-precipitate of aluminum hydroxide and magnesium hydroxide, co-precipitate of aluminum hydroxide, magnesium carbonate and calcium carbonate, and co-precipitate of aluminum hydroxide and sodium hydrogen carbonate. Among these compounds, magnesium hydroxide is particularly preferable from the viewpoint of the disintegrating property and dissolution property of a preparation.

In another embodiment, the chemical stabilizer may comprise a basic salt, such as sodium phosphate dibasic or sodium phosphate tribasic.

Chemical stablizers according to the invention may be used alone or in combination of two or more. Metal oxides and/or metal hydroxides used as chemical stabilers may be compounded in such an amount that they are quickly dissolved and neutralize gastric acid simultaneously with disintegration of a solid preparation in stomach, preferably, prior to dissolution of an active ingredient, in order to prevent unstabilization of substantial parts of an active ingredient by being exposed to gastric acid.

In an embodiment, the chemical stabilizer may be compounded in an amount of about 0.001 to 2000 parts by weight, preferably about 0.002 to 1000 parts by weight, more preferably about 0.002 to 100, still more preferably from about 0.002 to 10 parts by weight relative to 1 part by weight of a benzimidazole derivative.

It will be appreciated the dosage forms described herein, particularly in FIGS. 1-3 are merely exemplary of a variety of dosage forms designed for and capable of achieving administration of the inventive substance(s). Those of skill in the art can identify other dosage forms that would be suitable.

### IV. Methods of Administration

The inventive methods, compositions, and dosage forms are useful in treating a variety of indications that are treatable using benzimidazole derivatives. In an aspect, the invention provides a method for treating an indication, such as a disease or disorder, in a patient by administering an inventive composition or dosage form that comprises one or more benzimidazole derivatives. In an embodiment, a composition or dosage form comprising one or more benzimidazole derivatives is administered to the patient via oral administration. The dose administered is generally adjusted in accord with the age, weight, and condition of the patient, taking into consideration the dosage form and the desired result. Inventive dosage forms may comprise one or more benzimidazole derivatives in combination.

While there has been described and pointed out features and advantages of the invention, as applied to present embodiments, those skilled in the art will appreciate that various modifications, changes, additions, and omissions in the method described in the specification can be made without departing from the spirit of the invention. The following examples are intended to illustrate, and in no way limit, the scope of the present invention.

### V. Examples

### Example 1

### Oral Dosage Form

An oral dosage form is manufactured as follows: 1487 g of rabeprazole sodium, 5128 g of polyethylene oxide with average molecular weight of 900,000 or higher 2000 g of sodium chloride, USP and 2198 g of maltodextrin are added to a fluid bed granulator bowl. Next a binder solution is prepared by dissolving 400 g of hydroxypropylmethyl cellulose identified as 2910 having an average viscosity of 5 cps in 7,600 g of pH adjusted water The dry materials are fluid bed granulated by spraying with 4,000 g of binder solution. Next, the wet granulation is dried in the granulator to an acceptable moisture content, and sized using by passing through a 7-mesh screen. Next, the granulation is transferred to a blender and mixed with 5 g of butylated hydroxytoluene as an antioxidant and lubricated with 50 g of stearic acid.

Next, a second drug compartment composition is prepared as follows: 4123 g of rabeprazole sodium, 6416 g of polyethylene oxide with average molecular weight of 900,000 and 2750 g of maltodextrin are added to a fluid bed granulator bowl. Next a binder solution is prepared by dissolving 400 g of hydroxypropylmethyl cellulose identified as 2910 having an average viscosity of 5 cps in 7,600 g of water. The dry materials are fluid bed granulated by spraying with 4,000 g of binder solution. Next, the wet granulation is dried in the granulator to an acceptable moisture content, and sized using by passing through a 7-mesh screen. Next, the granulation is transferred to a blender and mixed with 5 g of butylated hydroxytoluene as an antioxidant and lubricated with 50 g of sodium stearyl fumarate.

Next, a push composition is prepared as follows: first, a binder solution is prepared. 15.6 kg of polyvinylpyrrolidone identified as K29-32 having an average molecular weight of 40,000 is dissolved in 104.4 kg of water. Then, 24 kg of sodium chloride and 1.2 kg of ferric oxide are sized using a Quadro Comil with a 21-mesh screen. Then, the screened materials and 88.44 kg of Polyethylene oxide (approximately 7,000,000 molecular weight) are added to a fluid bed granulator bowl. The dry materials are fluidized and mixed while 46.2 kg of binder solution is sprayed from 3 nozzles onto the powder. The granulation is dried in the fluid-bed chamber to an acceptable moisture level. The coated granules are sized using a Fluid Air mill with a 7-mesh screen. The granulation is transferred to a tote tumbler, mixed with 15 g of butylated hydroxytoluene and lubricated with 294 g sodium stearyl fumarate.

Next, the rabeprazole sodium drug compositions for the first and the second compartments, followed by a barrier layer composed of ethylcellulose, and the push composition are compressed into trilayer tablets. First, 100 mg of the rabeprazole sodium compartment one composition is added to the die cavity and pre-compressed, then 300 mg of the rabeprazole sodium compartment two composition is added to the die cavity and pre-compressed, then 400 mg of the push composition is added and the layers are pressed into a 9/32″ diameter longitudinal, deep concave, trilayer arrangement.

The trilayered arrangements are coated with a subcoat laminate. The wall forming composition comprises 70% hydroxypropyl cellulose identified as EF, having an average molecular weight of 80,000 and 30% of polyvinylpyrrolidone identified as K29-32 having an average molecular weight of 40,000. The wall-forming composition is dissolved in anhydrous ethyl alcohol, to make an 8% solids solution. The wall-forming composition is sprayed onto and around the bilayered arrangements in a pan coater until approximately 20 mg of laminate is applied to each tablet.

The trilayered arrangements are coated with a semi-permeable wall. The wall forming composition comprises 99% cellulose acetate having a 39.8% acetyl content and 1% polyethylene glycol comprising a 3.350 viscosity-average molecular weight. The wall-forming composition is dissolved in an acetone:water (95:5 wt:wt) co-solvent to make a 5% solids solution. The wall-forming composition is sprayed onto and around the bilayered arrangements in a pan coater until approximately 40 mg of membrane is applied to each tablet.

Next, two 25 mil (0.6 mm) exit passageways are laser drilled through the semi-permeable wall to connect the drug layer with the exterior of the dosage system. The residual solvent is removed by drying for 144 hours as 45 Deg C. and controlled humidity. After drilling, the osmotic systems are dried for 4 hours at 45 Deg C. to remove excess moisture.

### Example 2

### Oral Dosage Form

A matrix dosage form according to the present invention is prepared as follows. 8 grams of rabeprazole sodium, 25 grams of hydroxypropyl methylcellulose having a number average molecular weight of 9,200 grams per mole, and 15 grams of hydroxypropyl methylcellulose having a molecular weight of 242,000 grams per mole, are passed through a screen having a mesh size of 40 wires per inch. The celluloses each have an average hydroxyl content of 8 weight percent and an average methoxyl content of 22 weight percent. The resulting sized powders are tumble mixed. Anhydrous ethyl alcohol is added slowly to the mixed powders with stirring until a dough consistency is produced. The damp mass is then extruded through a 20 mesh screen and dried overnight. The resulting dried material is re-screened through a 20 mesh screen to form the final granules. 2 grams of the tableting lubricant, sodium stearyl fumarate, and 15 g of butylated hydroxytoluene which are sized through an 80 mesh screen, are then tumbled into the granules.

625 mg of the resulting granulation is placed in a die cavity having an inside diameter of 9/32 inch and compressed with deep concave punch tooling. Each core contains a unit dose of rabeprazole sodium of 100 mg.

Next, the core is coated with an enteric coating. The enteric coating composition comprises 97% of hydroxypropylmethylcellulose phthalate 55S and 3% of triethylcitrate. The enteric coating composition is dissolved in 50/50 acetone/methanol mixture. The enteric coating composition is sprayed onto and around the capsules in a pan coater until approximately 40 mg of enteric coat is applied to each capsule. The residual solvent is removed by drying for 144 hours at 45 Deg C.

### Example 3

### RingCap Dosage Form with Enteric Coating

First, the cores of Example 2 is provided. Prior to coating with an enteric coating, rings of polyethylene having an inside diameter of 9/32 inch, a wall thickness of 0.013 inch, and a width of 2 mm are fabricated. These rings, or bands, are press fitted onto the cores of Example 2. The banded cores are then coated with the enteric coating of Example 2 and dried to complete the dosage form.

### Example 4

### Colonic Intubation Study

A single-dose, open-label, partially randomized, five-treatment, five-period, crossover pilot study was performed in healthy males to determine relative pharmacokinetics of rabeprazole, and comparators when dosed either orally or colonically. The study was a single dose study with blood sampling up to 24 hours following initiation.

Treatments A and B were rabeprazole sodium solution dosed to provide 30 and 60 mg, respectively, over 20 hours in the transverse colon of the subjects. Colonic dosing occurred using a nasoenteral tube. Treatment C was a single 20 mg dose of rabeprazole sodium oral enteric coated tablets dosed orally. Treatment D was placebo (200 mL of water) dosed orally. Treatment E was a single 40 mg dose of esomeprazole magnesium trihydrate dosed as an oral film-coated tablet.

The following rabeprazole and esomeprazole pharmacokinetic (PK) parameters were determined: maximum plasma concentration [Cₘₐₓ], time to maximum concentration [Tₘₐₓ], apparent elimination rate constant [k] and half-life [t_{1/2}], area under the plasma concentration-time profile (AUC) to time t [AUCₜ], where t is the time of the last quantifiable plasma concentration, and AUC to infinity [AUC_{inf}]. For subjects whose apparent elimination rate constant [k] could not be properly determined, the AUC to infinity [AUC_{inf}]) was calculated based on the median apparent elimination constant [k] of the subjects with available t_{1/2} values in the same treatment period. The lower limit of quantitation for rabeprazole and esomeprazole in human plasma was estimated to be about 5 ng/mL. The intragastric pH profile were determined.

Plasma rabeprazole and esomeprazole concentrations were obtained for the entire treatment period for all subjects. Tables A & B summarizes PK results for the 19 subjects that completed all 5 treatments and with available PK/PD data and the subset of 14 subjects that had “optimal” evaluable PK/PD data.

TABLE A
Mean (SD) PK Parameters for Rabeprazole and Esomeprazole (N = 19)

| | TRT A | | TRT B | | | | | |
|---|---|---|---|---|---|---|---|---|
| | Rabeprazole | | Rabeprazole | | TRT C | | TRT E | |
| | 30 mg solution in | | 60 mg solution | | Rabeprazole | | Esomeprazole | |
| Parameter | the colon | | in the colon | | 20 mg oral tablet | | 40 mg oral tablet | |
| Cmax (ng/mL) | 18.0 | (14.8) | 38.4 | (36.3) | 239.1 | (160.6) | 726.6 | (438.8) |
| Tmax (h)^{a} | 18.0 | (5.5) | 18.0 | (4.6) | 4.5 | (4.2) | 1.5 | (1.6) |
| t1/2 (h)^{a} | 2.0 | (1.2) | 1.9 | (1.8) | 0.96 | (0.3) | 0.8 | (0.6) |
| AUCt (ng · h/mL) | 287.4 | (229.7) | 548.8 | (526.8) | 533.8 | (369.0) | 1865.1 | (1607.2) |
| AUCinf (ng · h/mL) | 331.9 | (231.7) | 599.4 | (536.0) | 602.8 | (343.8) | 1909.9 | (1608.4) |

^{a}Median Tmax and t1/2 are presented.

TABLE B
Mean (SD) PK Parameters for Rabeprazole and Esomeprazole (N = 14)

| | TRT A | | TRT B | | | | | |
|---|---|---|---|---|---|---|---|---|
| | Rabeprazole | | Rabeprazole | | TRT C | | TRT E | |
| | 30 mg solution in | | 60 mg solution | | Rabeprazole | | Esomeprazole | |
| Parameter | the colon | | in the colon | | 20 mg oral tablet | | 40 mg oral tablet | |
| Cmax (ng/mL) | 18.7 | (15.0) | 43.7 | (39.6) | 266.7 | (161.2) | 800.0 | (380.5) |
| Tmax (h)^{a} | 18.0 | (5.5) | 18.0 | (4.9) | 6.0 | (4.5) | 1.5 | (1.5) |
| t1/2 (h)^{a} | 2.0 | (1.2) | 2.1 | (1.9) | 1.0 | (0.1) | 0.9 | (0.6) |
| AUCt (ng · h/mL) | 289.6 | (216.4) | 585.5 | (582.8) | 618.7 | (374.7) | 2274.5 | (1668.5) |
| AUCinf (ng · h/mL) | 342.7 | (214.5) | 645.9 | (590.6) | 696.9 | (337.5) | 2310.7 | (1682.7) |

^{a}Median Tmax and t1/2 are presented.

Mean plasma concentration-time profiles for rabeprazole and esomeprazole are presented in FIG. 4.

The median Tₘₐₓ was approximately 18 hours after the start of treatment (colon infusion ended at 20 hr) for both 30 mg and 60 mg rabeprazole colonic solution treatment, compared to 4.5 to 6 hr for the 20 mg rabeprazole oral tablet treatment. The Cmax values were 18.0 and 38.4 ng/mL, respectively for the 30 mg and 60 mg rabeprazole colonic treatment, compared to a mean Cmax value of 239.1 ng/ml for the 20 mg tablet treatment. The median plasma half-life of rabeprazole was approximately 1 hour following oral administration and 2 hours following colonic treatment. Rabeprazole mean Cmax and AUC increased approximately dose proportionally following the 30 mg and 60 mg colonic treatments. A comparable AUC was achieved from the 60 mg colonic treatment and 20 mg oral tablet with lower Cmax values following the colonic treatment. The percent relative bioavailability was approximately 40% for the colonic treatment compared to that of the oral.

After oral administration of 40 mg esomeprazole, peak plasma concentration occurred around 1.5 hour with a mean Cmax value of 800 ng/mL. The median half-life was approximately 1 hour.

The intragastric pH profile was analyzed based on the percentage of time that the intragastric pH was above 4, which was calculated for the full 24 hours. In addition, the mean pH value over 5 time intervals of 6 hours each during the 30-hr sampling time was also estimated. The pharmacokinetic (PD) data is summarized below for the 19 subjects that completed all 5 treatments and with available PK/PD data and the subset of 14 subjects that had “optimal” evaluable PK/PD data.

The mean intragastric pH profiles for all of the treatments are shown in FIG. 4. Similar to what has been reported in the literature, gastric acid secretion was highest with lowest gastric pH occurring at night (corresponding to 12 and 20 hour after the start of dosing, and the clock time from 10 pm to 6 am). In addition, an increase in intragastric pH was noted after each meal (FIG. 4). Table C shows the mean percentage of time with pH >4 ranges from 6 to 27% with placebo treatment. Due to large variability in Tmax following treatment with rabeprazole 20 mg tablet, the mean concentration profile shown in FIG. 4 is not representative of the profile seen in individual subjects.

The median Tₘₐₓ was approximately 18 hours after the start of treatment (colon infusion ended at 20 hr) for both 30 mg and 60 mg rabeprazole colonic solution treatment, compared to 4.5 to 6 hr for the 20 mg rabeprazole oral tablet treatment. The Cmax values were 18.0 and 38.4 ng/mL, respectively for the 30 mg and 60 mg rabeprazole colonic treatment, compared to a mean Cmax value of 239.1 ng/ml for the 20 mg tablet treatment. The median plasma half-life of rabeprazole was approximately 1 hour following oral administration and 2 hours following colonic treatment. Rabeprazole mean Cmax and AUC increased approximately dose proportionally following the 30 mg and 60 mg colonic treatments. A comparable AUC was achieved from the 60 mg colonic treatment and 20 mg oral tablet with lower Cmax values following the colonic treatment.

After oral administration of 40 mg esomeprazole, peak plasma concentration occurred around 1.5 hour with a mean Cmax value of 800 ng/mL. The median half-life was approximately 1 hour.

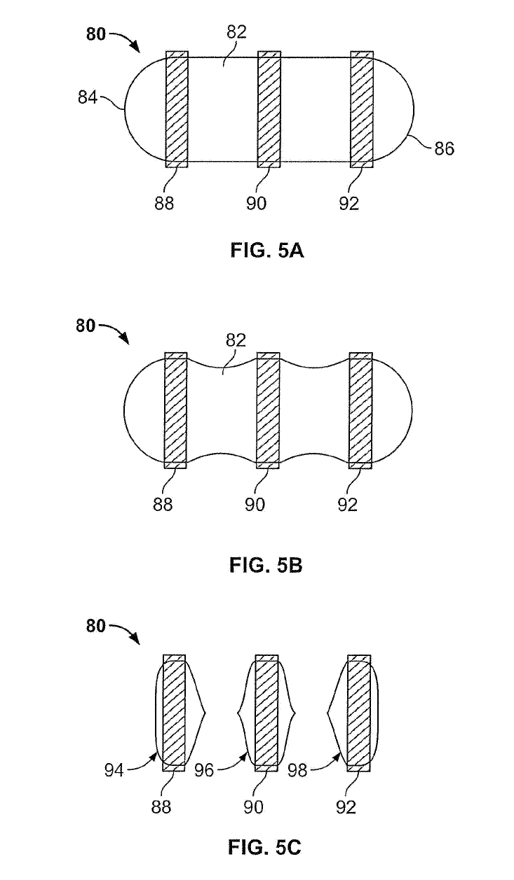

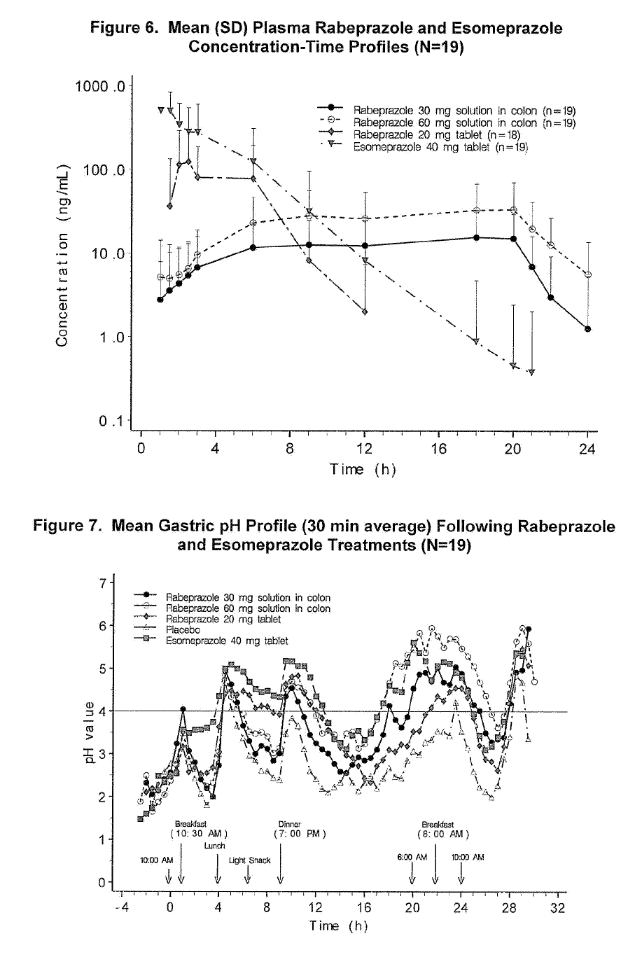

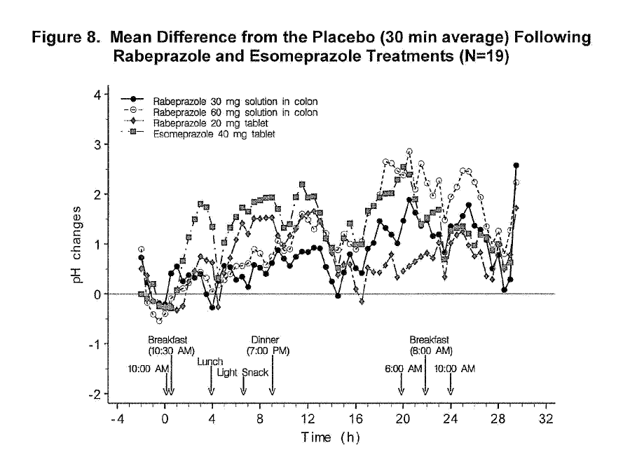

The mean intragastric pH profiles for all of the treatments are shown in FIG. 5. Similar to what has been reported in the literature, gastric acid secretion was highest with lowest gastric pH occurring at night (corresponding to 12 and 20 hour after the start of dosing, and the clock time from 10 pm to 6 am). In addition, an increase in intragastric pH was noted after each meal (FIGS. 5 & 6). Table C shows the mean percentage of time with pH >4 ranges from 6 to 27% with placebo treatment.

TABLE C
Mean (SD) percent of time with pH >4 following Rabeprazole,
Esomeprazole And Placebo treatments (N = 19)

| | TRT A | | TRT B | | TRT C | | | | TRT E | |
|---|---|---|---|---|---|---|---|---|---|---|
| | Rabeprazole | | Rabeprazole | | Rabeprazole | | TRT D | | Esomeprazole | |
| | 30 mg solution | | 60 mg solution | | 20 mg oral | | Water | | 40 mg oral | |
| Time (hr) | in the colon | | in the colon | | tablet | | Placebo | | tablet | |
| 0-6 (10 am-4 pm) | 30.6 | (14.0)^{b} | 32.6 | (21.6)^{b} | 31.1 | (17.9)^{b} | 27.0 | (14.1) | 57.0 | (22.9)^{a} |
| 6-12 (4 pm-10 pm) | 37.9 | (27.1)^{b,c} | 50.5 | (29.9)^{a,b} | 60.9 | (35.3)^{a} | 21.3 | (16.3) | 76.3 | (25.5)^{a} |
| 12-18 (10 pm-4 am) | 23.2 | (30.1) | 34.3 | (35.4)^{a} | 25.9 | (29.1) | 11.9 | (22.6) | 36.6 | (32.5)^{a} |
| 18-24 (4 am-10 am) | 47.2 | (34.6) | 64.0 | (38.9)^{a,c} | 37.8 | (34.5) | 27.6 | (26.5) | 56.0 | (31.3)^{a} |
| 24-30 (10 am-4 pm) | 45.4 | (29.6)^{a} | 64.4 | (27.6)^{a,c} | 43.6 | (19.6)^{a} | 22.6 | (12.8) | 49.5 | (23.0)^{a} |
| 24-hr average | 34.7 | (20.8)^{b} | 44.9 | (20.6)^{a} | 38.9 | (21.0)^{a,b} | 21.9 | (15.8)^{b} | 56.5 | (20.8)^{a} |

^{a}significantly different from placebo.
^{b}significantly different from Treatment E.
^{c}significantly different from Treatment C.

During the first 6 hours of treatment, the effects of the rabeprazole treatments (Treatments A, B and C) were not significantly different from placebo. In contrast, over the same time period, the esomeprazole treatment had a significant increase in the percentage of time with pH >4 compared to placebo.

This is consistent with a more rapid increase in concentration with Cmax achieved in about 1.5 h with the esomeprazole treatment (FIG. 4 and Table A).

Starting at the 6-12 hr time period and thereafter, the percentage of time with pH >4 with all four active treatments were significantly higher or a trend to be higher than placebo was noted, as shown in Table C. In addition, a dose-dependent increase in the percentage of time with pH >4 was observed between the 30 and 60 mg colonic rabeprazole treatments at the 6-12 h period and beyond as shown in Table C.

The 24-hr average percent of time with pH greater than 4 was significantly higher in all four active treatments compared to placebo and the order was 40 mg esomeprazole (57%) >60 mg rabeprazole colonic infusion (45%) >20 mg rabeprazole tablet (39%) >30 mg rabeprazole colonic infusion (35%), as shown in Table C.

A subset of the 14 subjects who had optimal evaluable PK/PD data were analyzed separately to be able to present a more optimal comparison of 60 mg rabeprazole colonic infusion versus the three reference treatments. In general, the trend is same for both sets of analysis (n=14 and 19).

TABLE D
Mean (SD) percent of time with pH >4 following Rabeprazole,
Esomeprazole And Placebo treatments (N = 14)

| | TRT B | | | | | | | |
|---|---|---|---|---|---|---|---|---|
| | Rabeprazole | | TRT C | | TRT D | | TRT E | |
| | 60 mg solution in | | Rabeprazole | | Water | | Esomeprazole | |
| Time (hr) | the colon | | 20 mg oral tablet | | Placebo | | 40 mg oral tablet | |
| 0-6 (10 am-4 pm) | 34.6 | (24.8)^{b} | 29.1 | (15.9)^{b} | 27.0 | (15.5) | 64.3 | (21.9)^{a} |
| 6-12 (4 pm-10 pm) | 55.8 | (28.4)^{a,b} | 63.6 | (36.7)^{a,b} | 22.5 | (17.1) | 87.0 | (17.4)^{a} |
| 12-18 (10 pm-4 am) | 39.9 | (37.2)^{a} | 26.7 | (28.8) | 6.7 | (15.4) | 42.8 | (34.5)^{a} |
| 18-24 (4 am-10 am) | 68.9 | (39.2)^{a,c} | 35.4 | (30.0) | 19.5 | (18.6) | 60.1 | (34.4)^{a} |
| 24-30 (10 am-4 pm) | 72.0 | (26.3)^{a,c} | 45.4 | (20.9)^{a} | 21.6 | (10.8) | 57.1 | (21.7)^{a} |
| 24-hr average | 49.2 | (19.7)^{a} | 38.7 | (19.9)^{a} | 18.9 | (13.1) | 63.5 | (19.3)^{a} |

^{a}significantly different from placebo.
^{b}significantly different from Treatment E.
^{c}significantly different from Treatment C.

What is claimed is:

1. A method comprising:
orally administering oral sustained release dosage forms comprising a benzimidazole derivative to a patient suffering from one or more gastric acid related diseases;
wherein the benzimidazole derivative is sustainably released from the oral sustained release dosage form for a period of at least about 10 hours, and at rates effective to maintain an intragastric pH of the patient ≧about 4 during at least about 65% of the period.

2. The method of claim 1, wherein the benzimidazole derivative comprises a pharmaceutically acceptable salt of rabeprazole.

3. The method of claim 2, wherein the pharmaceutically acceptable salt of rabeprazole comprises rabeprazole sodium.

4. The method of claim 1, wherein the oral sustained release dosage form comprises an osmotic oral sustained release dosage form.

5. The method of claim 1, wherein the benzimidazole derivative is sustainably released from the oral sustained release dosage form for a period of at least about 12 hours.

6. The method of claim 5, wherein the benzimidazole derivative is sustainably released from the oral sustained release dosage form for a period of at least about 14 hours.

7. The method of claim 6, wherein the benzimidazole derivative is sustainably released from the oral sustained release dosage form for a period of at least about 16 hours.

8. The method of claim 7, wherein the benzimidazole derivative is sustainably released from the oral sustained release dosage form for a period of at least about 17 hours.

9. The method of claim 8, wherein the benzimidazole derivative is sustainably released from the oral sustained release dosage form for a period of at least about 18 hours.

10. The method of claim 1, wherein the benzimidazole derivative is sustainably released from the oral sustained release dosage form at rates effective to maintain an intragastric pH of the patient ≧about 4 during at least about 70% of the period.

11. The method of claim 1, wherein the benzimidazole derivative is sustainably released from the oral sustained release dosage form at rates effective to maintain an intragastric pH of the patient ≧about 4 during at least about 75% of the period.

12. The method of claim 1, wherein the benzimidazole derivative is sustainably released from the oral sustained release dosage form at rates effective to maintain an intragastric pH of the patient ≧ about 4 during at least about 80% of the period.

13. The method of claim 1, wherein the gastric acid related indications comprise healing and/or maintenance of healing of erosive or ulcerative gastroesophogeal reflux disease

14. The method of claim 1, wherein the gastric acid related indications comprise healing of duodenal ulcers.

15. The method of claim 1, wherein the gastric acid related indications comprise treatment of pathological hypersecretory conditions;

16. The method of claim 1, wherein the pathological hypersecretory conditions comprise Zollinger-Ellison Syndrome.

17. The method of claim 1, wherein the gastric acid related indications comprise treatment of symptiomatic gastroesophogeal reflux disease.

18. The method of claim 1, wherein the gastric acid related indications comprise treatment of acid related extra esophageal manifestations of gastroesophogeal reflux disease in patients with chronic cough, asthma, COPD, pharyngitis, or otitis media.

19. The method of claim 1, wherein the gastric acid related indications comprise Heliobacter Pylori eradication to reduce risk of duodenal ulcer recurrence.
